SEQUENCE LISTING (15 sequences)

SEQ ID NO: 1 - DNA, 1644 bp, Lactococcus lactis
  CDS (1)..(1644): wild type kdcA
atgtatacagtaggagattacctgttagaccgattacacgagttgggaattgaagaaatttttggagttcctggtgactataacttacaatttttagatcaaattatttcacgcgaagatatgaaatggattggaaatgctaatgaattaaatgcttcttatatggctgatggttatgctcgtactaaaaaagctgccgcatttctcaccacatttggagtcggcgaattgagtgcgatcaatggactggcaggaagttatgccgaaaatttaccagtagtagaaattgttggttcaccaacttcaaaagtacaaaatgacggaaaatttgtccatcatacactagcagatggtgattttaaacactttatgaagatgcatgaacctgttacagcagcgcggactttactgacagcagaaaatgccacatatgaaattgaccgagtactttctcaattactaaaagaaagaaaaccagtctatattaacttaccagtcgatgttgctgcagcaaaagcagagaagcctgcattatctttagaaaaagaaagctctacaacaaatacaactgaacaagtgattttgagtaagattgaagaaagtttgaaaaatgcccaaaaaccagtagtgattgcaggacacgaagtaattagttttggtttagaaaaaacggtaactcagtttgtttcagaaacaaaactaccgattacgacactaaattttggtaaaagtgctgttgatgaatctttgccctcatttttaggaatatataacgggaaactttcagaaatcagtcttaaaaattttgtggagtccgcagactttatcctaatgcttggagtgaagcttacggactcctcaacaggtgcattcacacatcatttagatgaaaataaaatgatttcactaaacatagatgaaggaataattttcaataaagtggtagaagattttgattttagagcagtggtttcttctttatcagaattaaaaggaatagaatatgaaggacaatatattgataagcaatatgaagaatttattccatcaagtgctcccttatcacaagaccgtctatggcaggcagttgaaagtttgactcaaagcaatgaaacaatcgttgctgaacaaggaacctcattttttggagcttcaacaattttcttaaaatcaaatagtcgttttattggacaacctttatggggttctattggatatacttttccagcggctttaggaagccaaattgcggataaagagagcagacaccttttatttattggtgatggttcacttcaacttaccgtacaagaattaggactatcaatcagagaaaaactcaatccaatttgttttatcataaataatgatggttatacagttgaaagagaaatccacggacctactcaaagttataacgacattccaatgtggaattactcgaaattaccagaaacatttggagcaacagaagatcgtgtagtatcaaaaattgttagaacagagaatgaatttgtgtctgtcatgaaagaagcccaagcagatgtcaatagaatgtattggatagaactagttttggaaaaagaagatgcgccaaaattactgaaaaaaatgggtaaattatttgctgagcaaaataaatag

SEQ ID NO: 2 - protein, 547 aa, Lactococcus lactis
MYTVGDYLLDRLHELGIEEIFGVPGDYNLQFLDQIISREDMKWIGNANELNASYMADGYARTKKAAAFLTTFGVGELSAINGLAGSYAENLPVVEIVGSPTSKVQNDGKFVHHTLADGDFKHFMKMHEPVTAARTLLTAENATYEIDRVLSQLLKERKPVYINLPVDVAAAKAEKPALSLEKESSTTNTTEQVILSKIEESLKNAQKPVVIAGHEVISFGLEKTVTQFVSETKLPITTLNFGKSAVDESLPSFLGIYNGKLSEISLKNFVESADFILMLGVKLTDSSTGAFTHHLDENKMISLNIDEGIIFNKVVEDFDFRAVVSSLSELKGIEYEGQYIDKQYEEFIPSSAPLSQDRLWQAVESLTQSNETIVAEQGTSFFGASTIFLKSNSRFIGQPLWGSIGYTFPAALGSQIADKESRHLLFIGDGSLQLTVQELGLSIREKLNPICFIINNDGYTVEREIHGPTQSYNDIPMWNYSKLPETFGATEDRVVSKIVRTENEFVSVMKEAQADVNRMYWIELVLEKEDAPKLLKKMGKLFAEQNK

SEQ ID NO: 3 - DNA, 1644 bp, Artificial Sequence
  codon optimize gene for SEQ ID NO1
atgtatactgttggtgattatctgctggaccgtctgcatgaactgggcattgaagaaatcttcggtgtcccaggcgactacaacctgcagttcctggaccagatcatctcccgcgaagatatgaaatggatcggtaacgcaaacgagctgaacgcgtcttatatggctgatggttatgctcgcaccaaaaaggctgcggcctttctgaccacctttggtgtgggcgagctgagcgcgatcaacggcctggcaggttcctacgctgagaacctgccggtagtagaaatcgttggttccccgacctctaaggttcagaacgacggcaaattcgtacatcacaccctggcggacggcgattttaagcactttatgaaaatgcacgaaccggtcaccgccgctcgcactctgctgaccgcggaaaacgcaacgtacgagatcgatcgtgtactgtcccagctgctgaaagaacgtaaaccggtgtatatcaatctgccggttgatgtcgctgcggccaaagcagagaaaccggcactgtccctggagaaggagagctccactactaacaccaccgaacaggttatcctgtccaaaattgaagaatctctgaaaaacgcacagaaaccggtggttatcgcaggtcacgaggttatctccttcggcctggagaaaactgttactcaattcgtctctgaaacgaaactgccgatcacgaccctgaactttggcaagtccgcagttgacgaatctctgccttctttcctgggcatttacaacggcaaactgtccgagatctccctgaagaacttcgtagaatccgctgactttatcctgatgctgggtgtgaaactgaccgactcctctaccggtgcgttcacgcaccatctggatgaaaacaaaatgatcagcctgaacatcgacgagggtatcatcttcaacaaggtagttgaagatttcgacttccgtgctgttgtcagcagcctgtccgagctgaaaggcattgagtacgagggtcaatacatcgataaacagtacgaagagtttattccgtcttctgcaccgctgagccaggaccgcctgtggcaggcagttgagtccctgacgcagtccaacgaaactatcgtagcggaacaaggtacctctttcttcggtgcttctaccatctttctgaagtccaactctcgctttatcggtcagccgctgtggggttctatcggttacacgttcccggctgcgctgggtagccagatcgctgataaagagtctcgtcatctgctgttcatcggtgatggttccctgcagctgactgtacaggaactgggtctgtctatccgtgaaaaactgaacccgatttgttttatcatcaataacgatggctacactgttgagcgtgaaattcatggtccgactcagtcttacaacgatattccgatgtggaactactctaaactgccggaaaccttcggtgcaactgaggatcgcgtcgtgagcaagattgtgcgtactgagaacgagttcgtatctgttatgaaagaggcgcaggcagatgtgaaccgcatgtactggatcgaactggttctggaaaaagaggatgcaccgaaactgctgaagaaaatgggtaaactgtttgcggagcagaacaagtaa

SEQ ID NO: 4 - protein, 547 aa, Artificial Sequence
  synthetic alpha-ketopimelic acid decarboxylase
MYTVGDYLLDRLHELGIEEIFGVPGDYNLQFLDQIISREDMKWIGNANELNASYMADGYARTKKAAAFLTTXGVGELSAINGLAGSYAENLPVVEIVGSPXSKXQNDGKFXHHTLADGDFKHFMKMHEPVTAARTLLTAENATYEIDRVLSQLLKERKPVYINLPXDVAAAKAEKPALSLEKESSTTNTTEQVILSKIEESLKNAQKPVVIAGHEVISFGLEKTVTQFVSETKLPITTLXXGKSAVDESLPSFLGIYXGKXSEISLKNFVESADFILMLGVKLXDSSTGXXTHHLDENKMISLNIDEGIIFNKVVEDFDFRAVVSSLSELKGIEYEGQYIDKQYEEFIPSSAPLSQDRLWQAVESLTQSNETIVAEXGTSXXGASTIFLKSNSRFIGQPLWGSIGYTFPAALGSQIADKESRHLLFIGDGSLQLTVQELGLSIREKLNPICFIINNDGYTXEREXHGPTQSYNDIPMWNYSKLPETFGATEDRVVSKIVRTENEFVSVMKEAQADVNRMYWIELVLEKEDAXKXXKKXXKXXAEXXX

SEQ ID NO: 5 - protein, 547 aa, Artificial Sequence; identical to SEQ ID NO: 2
  synthetic alpha-ketopimelic acid decarboxylase mutations

SEQ ID NO: 6 - DNA, 5750 bp, Artificial Sequence
  pBAD_DEST_kdcA
aagaaaccaattgtccatattgcatcagacattgccgtcactgcgtcttttactggctcttctcgctaaccaaaccggtaaccccgcttattaaaagcattctgtaacaaagcgggaccaaagccatgacaaaaacgcgtaacaaaagtgtctataatcacggcagaaaagtccacattgattatttgcacggcgtcacactttgctatgccatagcatttttatccataagattagcggatcctacctgacgctttttatcgcaactctctactgtttctccatacccgttttttgggctaacacaagtttgtacaaaaaagcaggctaggaggaattacatatgtatactgttggtgattatctgctggaccgtctgcatgaactgggcattgaagaaatcttcggtgtcccaggcgactacaacctgcagttcctggaccagatcatctcccgcgaagatatgaaatggatcggtaacgcaaacgagctgaacgcgtcttatatggctgatggttatgctcgcaccaaaaaggctgcggcctttctgaccacctttggtgtgggcgagctgagcgcgatcaacggcctggcaggttcctacgctgagaacctgccggtagtagaaatcgttggttccccgacctctaaggttcagaacgacggcaaattcgtacatcacaccctggcggacggcgattttaagcactttatgaaaatgcacgaaccggtcaccgccgctcgcactctgctgaccgcggaaaacgcaacgtacgagatcgatcgtgtactgtcccagctgctgaaagaacgtaaaccggtgtatatcaatctgccggttgatgtcgctgcggccaaagcagagaaaccggcactgtccctggagaaggagagctccactactaacaccaccgaacaggttatcctgtccaaaattgaagaatctctgaaaaacgcacagaaaccggtggttatcgcaggtcacgaggttatctccttcggcctggagaaaactgttactcaattcgtctctgaaacgaaactgccgatcacgaccctgaactttggcaagtccgcagttgacgaatctctgccttctttcctgggcatttacaacggcaaactgtccgagatctccctgaagaacttcgtagaatccgctgactttatcctgatgctgggtgtgaaactgaccgactcctctaccggtgcgttcacgcaccatctggatgaaaacaaaatgatcagcctgaacatcgacgagggtatcatcttcaacaaggtagttgaagatttcgacttccgtgctgttgtcagcagcctgtccgagctgaaaggcattgagtacgagggtcaatacatcgataaacagtacgaagagtttattccgtcttctgcaccgctgagccaggaccgcctgtggcaggcagttgagtccctgacgcagtccaacgaaactatcgtagcggaacaaggtacctctttcttcggtgcttctaccatctttctgaagtccaactctcgctttatcggtcagccgctgtggggttctatcggttacacgttcccggctgcgctgggtagccagatcgctgataaagagtctcgtcatctgctgttcatcggtgatggttccctgcagctgactgtacaggaactgggtctgtctatccgtgaaaaactgaacccgatttgttttatcatcaataacgatggctacactgttgagcgtgaaattcatggtccgactcagtcttacaacgatattccgatgtggaactactctaaactgccggaaaccttcggtgcaactgaggatcgcgtcgtgagcaagattgtgcgtactgagaacgagttcgtatctgttatgaaagaggcgcaggcagatgtgaaccgcatgtactggatcgaactggttctggaaaaagaggatgcaccgaaactgctgaagaaaatgggtaaactgtttgcggagcagaacaagtaataagcttcccgggacccagctttcttgtacaaagtggttacgtagaacaaaaactcatctcagaagaggatctgaatagcgccgtcgaccatcatcatcatcatcattgagtttaaacggtctccagcttggctgttttggcggatgagagaagattttcagcctgatacagattaaatcagaacgcagaagcggtctgataaaacagaatttgcctggcggcagtagcgcggtggtcccacctgaccccatgccgaactcagaagtgaaacgccgtagcgccgatggtagtgtggggtctccccatgcgagagtagggaactgccaggcatcaaataaaacgaaaggctcagtcgaaagactgggcctttcgttttatctgttgtttgtcggtgaacgctctcctgagtaggacaaatccgccgggagcggatttgaacgttgcgaagcaacggcccggagggtggcgggcaggacgcccgccataaactgccaggcatcaaattaagcagaaggccatcctgacggatggcctttttgcgtttctacaaactctttttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagcagatcaattcgcgcgcgaaggcgaagcggcatgcataatgtgcctgtcaaatggacgaagcagggattctgcaaaccctatgctactccgtcaagccgtcaattgtctgattcgttaccaattatgacaacttgacggctacatcattcactttttcttcacaaccggcacggaactcgctcgggctggccccggtgcattttttaaatacccgcgagaaatagagttgatcgtcaaaaccaacattgcgaccgacggtggcgataggcatccgggtggtgctcaaaagcagcttcgcctggctgatacgttggtcctcgcgccagcttaagacgctaatccctaactgctggcggaaaagatgtgacagacgcgacggcgacaagcaaacatgctgtgcgacgctggcgatatcaaaattgctgtctgccaggtgatcgctgatgtactgacaagcctcgcgtacccgattatccatcggtggatggagcgactcgttaatcgcttccatgcgccgcagtaacaattgctcaagcagatttatcgccagcagctccgaatagcgcccttccccttgcccggcgttaatgatttgcccaaacaggtcgctgaaatgcggctggtgcgcttcatccgggcgaaagaaccccgtattggcaaatattgacggccagttaagccattcatgccagtaggcgcgcggacgaaagtaaacccactggtgataccattcgcgagcctccggatgacgaccgtagtgatgaatctctcctggcgggaacagcaaaatatcacccggtcggcaaacaaattctcgtccctgatttttcaccaccccctgaccgcgaatggtgagattgagaatataacctttcattcccagcggtcggtcgataaaaaaatcgagataaccgttggcctcaatcggcgttaaacccgccaccagatgggcattaaacgagtatcccggcagcaggggatcattttgcgcttcagccatacttttcatactcccgccattcagag

SEQ ID NO: 7 - DNA, 1032 bp, Artificial Sequence
  Methanococcus aeolicus codon optimized gene AksF for C.glutamicum, Maeo_1484
atgaagatccctaagatctgcgttatcgagggcgacggcatcggcaaggaagtcatcccagagactgttcgcatcctgaaggaaatcggtgacttcgagttcatctacgagcacgctggctacgagtgcttcaagcgctgtggcgacgcaatcccagaaaagaccctcaagaccgcaaaggaatgcgacgcaatcctgttcggtgctgtttccaccccaaagctggatgagactgagcgcaagccttacaagtccccaatcctcaccctgcgtaaggaactcgacctctacgcaaacgttcgcccaatccacaagctcgacaactccgattcctccaacaacatcgacttcatcatcatccgtgagaacaccgagggcctgtactccggtgttgagtactacgacgaggagaaggaactcgcaatctctgagcgccacatctccaagaagggctccaagcgcatcatcaagttcgctttcgagtacgcagtcaagcaccaccgcaagaaggtttcctgcatccacaagtccaacatcctgcgcatcaccgacggcctgttcctcaacatcttcaacgagttcaaggagaagtacaagaacgagtacaacatcgagggcaacgactacctggttgatgcaaccgcaatgtacatcctcaagtccccacagatgttcgacgtcatcgtcaccaccaacctgttcggcgacatcctgtccgatgaggcttccggcctgctcggtggccttggtcttgcaccttccgctaacatcggcgacaactacggcctgttcgagccagttcacggttccgctcctgacatcgctggcaagggcgttgcaaacccaatcgctgctgttctgtccgcatccatgatgctctactacctcgacatgaaggaaaagtcccgtctgctgaaggacgctgtcaagcaggttcttgctcacaaggacatcaccccagacctcggtggcaacctcaagaccaaggaagtttctgacaagatcatcgaagagctgcgtaagatctcgtaa

SEQ ID NO: 8 - DNA, 1155 bp, Artificial Sequence
  codon optimized nifV gene for C. glutamicum
atggcttccgtcatcatcgatgacaccaccctgcgcgacggcgagcagtccgctggtgttgcattcaacgctgatgagaagatcgcaatcgctcgcgcactggctgaactcggcgttcctgagcttgagatcggcatcccttccatgggtgaagaagagcgtgaggtcatgcacgcaatcgctggtcttggtctgtcctcacgcctcctcgcatggtgccgtctgtgcgacgttgacctcgcagctgcacgttccaccggtgtcaccatggttgacctctccctgccagtttctgacctcatgctgcaccacaagctcaaccgcgaccgcgactgggcactgcgtgaggttgctcgcctcgttggcgaggctcgcatggctggtcttgaggtctgcctcggctgcgaagatgcttcccgcgcagaccttgagttcgttgttcaggttggtgaagttgctcaggctgctggcgctcgccgcctgcgcttcgctgacaccgttggtgtcatggagccattcggcatgctcgaccgcttccgcttcctgtcccgtcgtctggacatggagcttgaggtccacgcacacgacgacttcggcctcgcaactgcaaacaccctggctgctgtcatgggtggcgcaacccacatcaacaccaccgtcaacggcctcggcgagcgcgcaggcaacgctgcactggaagagtgcgttctcgcactgaagaacctgcacggcatcgacaccggcatcgacacccgtggcatcccagcaatctccgcactggttgagcgcgcatccggccgtcaggttgcatggcagaagtccgttgttggtgctggcgttttcacccacgaggctggcatccacgttgacggcctgctgaagcaccgccgcaactacgaaggcctcaacccagatgagctgggccgctcccactccctggtcctcggcaagcactccggcgcacacatggttcgcaacacctaccgcgacctcggcatcgagctggctgactggcagtcccaggcactgctcggccgcatccgtgcattctccacccgcaccaagcgttccccacagcctgctgaactccaggacttctaccgccagctgtgtgagcagggcaacccagagctggcagctggtggcatggcctaa

SEQ ID NO: 9 - DNA, 9488 bp, Artificial Sequence
  pAKP-96 (vfl-kdcA (wt))
gcatacagcatggcctgcaacgcgggcatcccgatgccgccggaagcgagaagaatcataatggggaaggccatccagcctcgcgtcgcgaacgccagcaagacgtagcccagcgcgtcggccagcttgcaattcgcgctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgtggtttgccccagcaggcgaaaatcctgtttgatggtggttaacggcgggatataacatgagctgtcttcggtatcgtcgtatcccactaccgagatatccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcaccattcgatggtgtcaacgtaaatgccgcttcgccttcgcgcgcgaattgcaagctgatccgggcttatcgactgcacggtgcaccaatgcttctggcgtcaggcagccatcggaagctgtggtatggctgtgcaggtcgtaaatcactgcataattcgtgtcgctcaaggcgcactcccgttctggataatgttttttgcggccgcatcataacggttctggcaaatattctgaaatgagctgttgacaattaatcatcggctcgtataatgtgtggaattgtgagcggataacaatttcacacaggaaacagaattcgagctcggtaccgctaggaggaattaaccatgaataaaccacagtcttgggaagctcgtgctgaaacctatagcctgtacggctttaccgatatgccgtctctgcaccagcgtggtactgtagtggtaacgcacggtgagggcccgtacatcgtggacgttaatggccgccgttacctggatgcaaacagcggcctgtggaacatggttgcgggcttcgaccacaaaggcctgatcgatgccgcaaaagcgcagtacgaacgcttcccgggttatcacgcgttctttggccgtatgagcgaccagactgtgatgctgagcgaaaaactggttgaagtgtccccgttcgatagcggtcgtgtcttttacactaactctggcagcgaggctaacgataccatggttaagatgctgtggttcctgcacgcagcggaaggcaaacctcagaaacgtaaaattctgacccgttggaacgcttatcacggtgtgactgctgtttccgcatctatgaccggtaaaccgtataacagcgtgttcggtctgccgctgcctggcttcgtgcatctgacctgcccgcactactggcgttatggtgaggaaggcgaaactgaggaacagttcgtggcgcgtctggctcgtgaactggaagaaaccattcaacgcgaaggtgcagatactatcgcgggcttctttgcggagcctgttatgggtgccggcggtgtgattccgccggcgaagggctatttccaggcaatcctgccgatcctgcgcaagtacgacattccggttatttctgacgaagtgatctgcggcttcggccgcaccggtaacacctggggctgcgtgacgtatgacttcactccggacgcaatcattagctctaaaaacctgactgcgggtttcttccctatgggcgccgtaatcctgggcccagaactgtctaagcgcctggaaaccgccatcgaggcaatcgaagagttcccgcacggtttcactgctagcggccatccggtaggctgcgcaatcgcgctgaaggcgatcgatgttgtcatgaacgagggcctggcggaaaacgtgcgccgcctggcgccgcgttttgaagaacgtctgaaacacattgctgagcgcccgaacattggcgaatatcgcggcatcggtttcatgtgggccctggaagcagttaaagataaagctagcaagaccccgttcgacggcaacctgtccgtgagcgaacgtatcgctaatacctgtacggacctgggtctgatctgccgtccgctgggtcagtccgtagttctgtgcccaccatttatcctgaccgaagcgcagatggatgaaatgttcgataaactggagaaagctctggataaagtgttcgctgaagtcgcgtaaacccagcttactagtggctaggaggaattacatatgtatactgttggtgattatctgctggaccgtctgcatgaactgggcattgaagaaatcttcggtgtcccaggcgactacaacctgcagttcctggaccagatcatctcccgcgaagatatgaaatggatcggtaacgcaaacgagctgaacgcgtcttatatggctgatggttatgctcgcaccaaaaaggctgcggcctttctgaccacctttggtgtgggcgagctgagcgcgatcaacggcctggcaggttcctacgctgagaacctgccggtagtagaaatcgttggttccccgacctctaaggttcagaacgacggcaaattcgtacatcacaccctggcggacggcgattttaagcactttatgaaaatgcacgaaccggtcaccgccgctcgcactctgctgaccgcggaaaacgcaacgtacgagatcgatcgtgtactgtcccagctgctgaaagaacgtaaaccggtgtatatcaatctgccggttgatgtcgctgcggccaaagcagagaaaccggcactgtccctggagaaggagagctccactactaacaccaccgaacaggttatcctgtccaaaattgaagaatctctgaaaaacgcacagaaaccggtggttatcgcaggtcacgaggttatctccttcggcctggagaaaactgttactcaattcgtctctgaaacgaaactgccgatcacgaccctgaactttggcaagtccgcagttgacgaatctctgccttctttcctgggcatttacaacggcaaactgtccgagatctccctgaagaacttcgtagaatccgctgactttatcctgatgctgggtgtgaaactgaccgactcctctaccggtgcgttcacgcaccatctggatgaaaacaaaatgatcagcctgaacatcgacgagggtatcatcttcaacaaggtagttgaagatttcgacttccgtgctgttgtcagcagcctgtccgagctgaaaggcattgagtacgagggtcaatacatcgataaacagtacgaagagtttattccgtcttctgcaccgctgagccaggaccgcctgtggcaggcagttgagtccctgacgcagtccaacgaaactatcgtagcggaacaaggtacctctttcttcggtgcttctaccatctttctgaagtccaactctcgctttatcggtcagccgctgtggggttctatcggttacacgttcccggctgcgctgggtagccagatcgctgataaagagtctcgtcatctgctgttcatcggtgatggttccctgcagctgactgtacaggaactgggtctgtctatccgtgaaaaactgaacccgatttgttttatcatcaataacgatggctacactgttgagcgtgaaattcatggtccgactcagtcttacaacgatattccgatgtggaactactctaaactgccggaaaccttcggtgcaactgaggatcgcgtcgtgagcaagattgtgcgtactgagaacgagttcgtatctgttatgaaagaggcgcaggcagatgtgaaccgcatgtactggatcgaactggttctggaaaaagaggatgcaccgaaactgctgaagaaaatgggtaaactgtttgcggagcagaacaagtaataagcttctgttttggcggatgagagaagattttcagcctgatacagattaaatcagaacgcagaagcggtctgataaaacagaatttgcctggcggcagtagcgcggtggtcccacctgaccccatgccgaactcagaagtgaaacgccgtagcgccgatggtagtgtggggtctccccatgcgagagtagggaactgccaggcatcaaataaaacgaaaggctcagtcgaaagactgggcctttcgttttatctgttgtttgtcggtgaacgctctcctgagtaggacaaatccgccgggagcggatttgaacgttgcgaagcaacggcccggagggtggcgggcaggacgcccgccataaactgccaggcatcaaattaagcagaaggccatcctgacggatggcctttttgcgtttctacaaactcttttgtttatttttctaaatacattcaaatatgcggccgctcatgagacaataaccctgaccggtttattgactaccggaagcagtgtgaccgtgtgcttctcaaatgcctgaggccagtttgctcaggctctccccgtggaggtaataattgacgatatgatcatttattctgcctcccagagcctgataaaaacggtgaatccgttagcgaggtgccgccggcttccattcaggtcgaggtggcccggctccatgcaccgcgacgcaacgcggggaggcagacaaggtatagggcggcgaggcggctacagccgatagtctggaacagcgcacttacgggttgctgcgcaacccaagtgctaccggcgcggcagcgtgacccgtgtcggcggctccaacggctcgccatcgtccagaaaacacggctcatcgggcatcggcaggcgctgctgcccgcgccgttcccattcctccgtttcggtcaaggctggcaggtctggttccatgcccggaatgccgggctggctgggcggctcctcgccggggccggtcggtagttgctgctcgcccggatacagggtcgggatgcggcgcaggtcgccatgccccaacagcgattcgtcctggtcgtcgtgatcaaccaccacggcggcactgaacaccgacaggcgcaactggtcgcggggctggccccacgccacgcggtcattgaccacgtaggccgacacggtgccggggccgttgagcttcacgacggagatccagcgctcggccaccaagtccttgactgcgtattggaccgtccgcaaagaacgtccgatgagcttggaaagtgtcttctggctgaccaccacggcgttctggtggcccatctgcgccacgaggtgatgcagcagcattgccgccgtgggtttcctcgcaataagcccggcccacgcctcatgcgctttgcgttccgtttgcacccagtgaccgggcttgttcttggcttgaatgccgatttctctggactgcgtggccatgcttatctccatgcggtagggtgccgcacggttgcggcaccatgcgcaatcagctgcaacttttcggcagcgcgacaacaattatgcgttgcgtaaaagtggcagtcaattacagattttctttaacctacgcaatgagctattgcggggggtgccgcaatgagctgttgcgtaccccccttttttaagttgttgatttttaagtctttcgcatttcgccctatatctagttctttggtgcccaaagaagggcacccctgcggggttcccccacgccttcggcgcggctccccctccggcaaaaagtggcccctccggggcttgttgatcgactgcgcggccttcggccttgcccaaggtggcgctgcccccttggaacccccgcactcgccgccgtgaggctcggggggcaggcgggcgggcttcgccttcgactgcccccactcgcataggcttgggtcgttccaggcgcgtcaaggccaagccgctgcgcggtcgctgcgcgagccttgacccgccttccacttggtgtccaaccggcaagcgaagcgcgcaggccgcaggccggaggcttttccccagagaaaattaaaaaaattgatggggcaaggccgcaggccgcgcagttggagccggtgggtatgtggtcgaaggctgggtagccggtgggcaatccctgtggtcaagctcgtgggcaggcgcagcctgtccatcagcttgtccagcagggttgtccacgggccgagcgaagcgagccagccggtggccgctcgcggccatcgtccacatatccacgggctggcaagggagcgcagcgaccgcgcagggcgaagcccggagagcaagcccgtagggcgccgcagccgccgtaggcggtcacgactttgcgaagcaaagtctagtgagtatactcaagcattgagtggcccgccggaggcaccgccttgcgctgcccccgtcgagccggttggacaccaaaagggaggggcaggcatggcggcatacgcgatcatgcgatgcaagaagctggcgaaaatgggcaacgtggcggccagtctcaagcacgcctaccgcgagcgcgagacgcccaacgctgacgccagcaggacgccagagaacgagcactgggcggccagcagcaccgatgaagcgatgggccgactgcgcgagttgctgccagagaagcggcgcaaggacgctgtgttggcggtcgagtacgtcatgacggccagcccggaatggtggaagtcggccagccaagaacagcaggcggcgttcttcgagaaggcgcacaagtggctggcggacaagtacggggcggatcgcatcgtgacggccagcatccaccgtgacgaaaccagcccgcacatgaccgcgttcgtggtgccgctgacgcaggacggcaggctgtcggccaaggagttcatcggcaacaaagcgcagatgacccgcgaccagaccacgtttgcggccgctgtggccgatctagggctgcaacggggcatcgagggcagcaaggcacgtcacacgcgcattcaggcgttctacgaggccctggagcggccaccagtgggccacgtcaccatcagcccgcaagcggtcgagccacgcgcctatgcaccgcagggattggccgaaaagctgggaatctcaaagcgcgttgagacgccggaagccgtggccgaccggctgacaaaagcggttcggcaggggtatgagcctgccctacaggccgccgcaggagcgcgtgagatgcgcaagaaggccgatcaagcccaagagacggcccgagaccttcgggagcgcctgaagcccgttctggacgccctggggccgttgaatcgggatatgcaggccaaggccgccgcgatcatcaaggccgtgggcgaaaagctgctgacggaacagcgggaagtccagcgccagaaacaggcccagcgccagcaggaacgcgggcgcgcacatttccccgaaaagtgccacctgggatgaatgtcagctactgggctatctggacaagggaaaacgcaagcgcaaagagaaagcaggtagcttgcagtgggcttacatggcgatagctagactgggcggttttatggacagcaagcgaaccggaattgccagctggggcgccctctggtaaggttgggaagccctgcaaagtaaactggatggctttcttgccgccaaggatctgatggcgcaggggatcaagatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccacccccatgggcaaatattatacgcaaggcgacaaggtgctgatgccgctggcgattcaggttcatcatgccgtttgtgatggcttccatgtcggcagaatgcttaatgaattacaacagtttttat

SEQ ID NO: 10 - DNA, 8141 bp, Artificial Sequence
  pAKP-378
agcttggctgttttggcggatgagagaagattttcagcctgatacagattaaatcagaacgcagaagcggtctgataaaacagaatttgcctggcggcagtagcgcggtggtcccacctgaccccatgccgaactcagaagtgaaacgccgtagcgccgatggtagtgtggggtctccccatgcgagagtagggaactgccaggcatcaaataaaacgaaaggctcagtcgaaagactgggcctttcgttttatctgttgtttgtcggtgaacgctctcctgagtaggacaaatccgccgggagcggatttgaacgttgcgaagcaacggcccggagggtggcgggcaggacgcccgccataaactgccaggcatcaaattaagcagaaggccatcctgacggatggcctttttgcgtttctacaaactctttttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctncagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagcattgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcacgaacgccagcaagacgtagcccagcgcgtcggccagcttgcaattcgcgctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgctggtggttaacggcgggatataacatgagctgtcttcggtatcgtcgtatcccactaccgagatatccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcaccattcgatggtgtcaacgtaaatgccgcttcgccttcgcgcgcgaattgcaagctgatccgggcttatcgactgcacggtgcaccaatgcttctggcgtcaggcagccatcggaagctgtggtatggctgtgcaggtcgtaaatcactgcataattcgtgtcgctcaaggcgcactcccgttctggataatgttttttgcgccgacatcataacggttctggcaaatattctgaaatgagctgttgacaattaatcatcggctcgtataatgtgtggaattgtgagcggataacaatttcacacaggaaacagaattcgagctcggtacccggggatcctctagaaataattttgtttaactttaagaaggagatatacatatggctagcgtgatcatcgacgacactaccctgcgtgacggtgaacagagtgccggggtcgccttcaatgccgacgagaagatcgctatcgcccgcgcgctcgccgaactgggcgtgccggagttggagatcggcattcccagcatgggcgaggaagagcgcgaggtgatgcacgccatcgccggtctcggcctgtcgtctcgcctgctggcctggtgccggctatgcgacgtcgatctcgcggcggcgcgctccaccggggtgaccatggtcgacctttcgctgccggtctccgacctgatgctgcaccacaagctcaatcgcgatcgcgactgggccttgcgcgaagtggccaggctggtcggcgaagcgcgcatggccgggctcgaggtgtgcctgggctgcgaggacgcctcgcgggcggatctggagttcgtcgtgcaggtgggcgaagtggcgcaggccgccggcgcccgtcggctgcgcttcgccgacaccgtcggggtcatggagcccttcggcatgctcgaccgcttccgtttcctcagccggcgcctggacatggagctggaagtgcacgcccacgatgatttcgggctggccacggccaacaccctggccgcggtgatgggcggggcgactcatatcaacaccacggtcaacgggctcggcgagcgtgccggcaacgccgcgctggaagagtgcgtgctggcgctcaagaacctccacggtatcgacaccggtatcgatacccgcggcatcccggccatctccgcgctggtcgagcgggcctcggggcgccaggtggcctggcagaagagcgtggtcggcgccggggtgttcactcacgaggccggtatccacgtcgacggactgctcaagcatcggcgcaactacgaggggctgaatcccgacgaactcggtcgcagccacagtctggtgctgggcaagcattccggggcgcacatggtgcgcaacacgtaccgcgatctgggtatcgagctggcggactggcagagccaagcgctgctcggccgcatccgtgccttctccaccaggaccaagcgcagcccgcagcctgccgagctgcaggatttctatcggcagttgtgcgagcaaggcaatcccgaactggccgcaggaggaatggcatgataataaggtaccaggaggaaactataatgaagatcccgaaaatctgcgttatcgaaggtgacggtatcggtaaagaagttatcccagaaaccgttcgcattctgaaagaaatcggtgacttcgaattcatctacgaacacgctggttacgaatgcttcaagcgctgcggtgacgctatcccggagaaaactctgaaaactgcgaaagagtgcgacgctatcctgttcggtgcggtatctactccgaaactggacgaaactgaacgtaagccgtacaaatctccgattctgactctgcgtaaagaactggatctgtacgctaacgttcgtccgatccacaaactggataactctgactcctccaacaacatcgacttcatcatcatccgtgaaaacactgaaggtctgtactccggtgttgaatactacgacgaagaaaaagaactggcaatctctgaacgtcacatctccaagaaaggttccaagcgcatcatcaaattcgcattcgaatacgctgttaagcaccaccgtaagaaagtttcctgcatccacaagtctaacatcctgcgtatcactgacggtctgttcctgaacatcttcaacgaattcaaagaaaaatacaaaaacgaatacaacatcgaaggtaacgactacctggttgacgcaactgcgatgtacatcctgaaatctccgcagatgttcgacgttatcgttactaccaacctgttcggtgacattctgtctgacgaagcgtctggtctgctgggtggtctgggtctggcgccgtctgctaacatcggtgacaactacggtctgttcgaaccggttcacggttctgcaccggatatcgctggtaaaggcgttgctaacccgatcgctgcagtactgtctgcttctatgatgctgtactacctggatatgaaagagaagtctcgcctgctgaaagacgctgttaaacaggtactggcacacaaagacatcactccggacctgggtggtaacctgaaaaccaaagaagtttctgacaagatcatcgaagaactgcgtaagatctcgtaataaggtacctctagtcgcactcccgttctggataatgttttttgcgccgacatcataacggttctggcaaatattctgaaatgagctgttgacaattaatcatcggctcgtataatgtgtggaattgtgagcggataacaatttcacactctagaaggaggaattaaccatatgaacatcaccgagaagatcctgtctgctaaagcgaagaaagaagttactccgggtgaaatcatcgaaatcccggttgatctggcgatgtctcacgacggtacttctccgccagcaatcaaaactttcgaaaaagttgcgactaaagtatgggacaacgagaagattgctatcgtattcgaccacaacgtaccggctaacaccatcggttctgctgaattccagaaagtttgccgcgatttcatcaagaagcagaagatcaccaaaaactacatccacggtgacggtatctgccaccaggtactgccggaaaaaggtctggttgaaccgggtaaagttatcgttggtgctgactctcacacttgcacttacggtgcttacggcgcattctctaccggtatgggtgcgactgacctggcgatggtttacgcaactggtaaaacctggttcatggttccggaagctatcaagatggaagtttctggtgaactgaactcttacactgcaccgaaagacatcatcctgaaaatcatcggtgaagttggtattgctggcgcaacttacaaaactgcagaattctgcggtgaaaccattgagaagatgggcgtagaaggtcgtgcgactatctgcaacatggctatcgaaatgggtgcgaaaaacggtatcatggaaccgaacaaagaagttatccagtacgtttctcagcgtactggtaagaaagagtctgaactgaacatcgttaagtctgacgaagatgctcagtactctgaagaaatgcacttcgacatcactgacatggaaccgcagatcgcttgcccgaacgacgttgataacgttaaagacatctccaaagttgaaggtactgcggttgatcagtgcctgatcggttcctgcaccaacggtcgtctgtctgacctgaaagacgcttacgaaatcctgaaagacaacgaaatcaacaacgacactcgcctgctgattctgccggcatctgcagaaatctacaagcaggctatccacgaaggttacatcgacgcattcatcgacgctggtgctatcatctgcaacccaggttgcggtccgtgcctgggtggtcacatgggcgtactgtctgaaggtgaaacttgcctgtctaccactaaccgtaacttcaaaggtcgtatgggcgacccgaaatcttccgtttacctggctaactccaaagttgttgctgcatctgcaatcgaaggtgttatcactaacccgaaagacctgtaataaggtaccaggaggaattaaccatatggacatcatcaaaggtaaaacctggactttcggtgaaaacatcgacactgacgttatcatcccaggtcgttacctccgcactttcaacccgcaggacctggcagaccacgtactggaaggtgaacgtccggacttcaccaagaacgttaagaaaggcgacatcatcgttgctgacgaaaacttcggttgcggttcttctcgcgaacaggcaccggttgctatcaaaactgctggcgttgatgctatcgttgcgaagtctttcgcacgtatcttctaccgtaacgctatcaacatcggtctgccggttatcgtttgcgacattcaggcgaaagacggtgacatcatcaacatcgacctgtctaaaggtattctgactaacgaaaccactggcgaatccgtaactttcgaaccgttcaaagagttcatgctggatatcctggaagataacggtctggttaaccactacctgaaagaaaaacagtaataacccggga

SEQ ID NO: 11 - DNA, 12495 bp, Artificial Sequence
  pAKP485
aagcttgcatgcctgcagaggaggaattaacatggcttccgtcatcatcgatgacaccaccctgcgcgacggcgagcagtccgctggtgttgcattcaacgctgatgagaagatcgcaatcgctcgcgcactggctgaactcggcgttcctgagcttgagatcggcatcccttccatgggtgaagaagagcgtgaggtcatgcacgcaatcgctggtcttggtctgtcctcacgcctcctcgcatggtgccgtctgtgcgacgttgacctcgcagctgcacgttccaccggtgtcaccatggttgacctctccctgccagtttctgacctcatgctgcaccacaagctcaaccgcgaccgcgactgggcactgcgtgaggttgctcgcctcgttggcgaggctcgcatggctggtcttgaggtctgcctcggctgcgaagatgcttcccgcgcagaccttgagttcgttgttcaggttggtgaagttgctcaggctgctggcgctcgccgcctgcgcttcgctgacaccgttggtgtcatggagccattcggcatgctcgaccgcttccgcttcctgtcccgtcgtctggacatggagcttgaggtccacgcacacgacgacttcggcctcgcaactgcaaacaccctggctgctgtcatgggtggcgcaacccacatcaacaccaccgtcaacggcctcggcgagcgcgcaggcaacgctgcactggaagagtgcgttctcgcactgaagaacctgcacggcatcgacaccggcatcgacacccgtggcatcccagcaatctccgcactggttgagcgcgcatccggccgtcaggttgcatggcagaagtccgttgttggtgctggcgttttcacccacgaggctggcatccacgttgacggcctgctgaagcaccgccgcaactacgaaggcctcaacccagatgagctgggccgctcccactccctggtcctcggcaagcactccggcgcacacatggttcgcaacacctaccgcgacctcggcatcgagctggctgactggcagtcccaggcactgctcggccgcatccgtgcattctccacccgcaccaagcgttccccacagcctgctgaactccaggacttctaccgccagctgtgtgagcagggcaacccagagctggcagctggtggcatggcctaataataatctagaaggaggaattaacatgaagatccctaagatctgcgttatcgagggcgacggcatcggcaaggaagtcatcccagagactgttcgcatcctgaaggaaatcggtgacttcgagttcatctacgagcacgctggctacgagtgcttcaagcgctgtggcgacgcaatcccagaaaagaccctcaagaccgcaaaggaatgcgacgcaatcctgttcggtgctgtttccaccccaaagctggatgagactgagcgcaagccttacaagtccccaatcctcaccctgcgtaaggaactcgacctctacgcaaacgttcgcccaatccacaagctcgacaactccgattcctccaacaacatcgacttcatcatcatccgtgagaacaccgagggcctgtactccggtgttgagtactacgacgaggagaaggaactcgcaatctctgagcgccacatctccaagaagggctccaagcgcatcatcaagttcgctttcgagtacgcagtcaagcaccaccgcaagaaggtttcctgcatccacaagtccaacatcctgcgcatcaccgacggcctgttcctcaacatcttcaacgagttcaaggagaagtacaagaacgagtacaacatcgagggcaacgactacctggttgatgcaaccgcaatgtacatcctcaagtccccacagatgttcgacgtcatcgtcaccaccaacctgttcggcgacatcctgtccgatgaggcttccggcctgctcggtggccttggtcttgcaccttccgctaacatcggcgacaactacggcctgttcgagccagttcacggttccgctcctgacatcgctggcaagggcgttgcaaacccaatcgctgctgttctgtccgcatccatgatgctctactacctcgacatgaaggaaaagtcccgtctgctgaaggacgctgtcaagcaggttcttgctcacaaggacatcaccccagacctcggtggcaacctcaagaccaaggaagtttctgacaagatcatcgaagagctgcgtaagatctcgtaataataaggatccactagtcgcactcccgttctggataatgttttttgcgccgacatcataacggttctggcaaatattctgaaatgagctgttgacaattaatcatcggctcgtataatgtgtggaattgtgagcggataacaatttcacactctagaaggaggaattaaccatatgactctggctgaagaaatcctgtccaagaaagttggtaagaaagttaaagcgggtgacgttgttgaaatcgatatcgacctggcgatgactcacgacggtactactccgctgtctgcgaaagcattcaagcagatcactgacaaagtatgggataacaagaaaatcgttatcgttttcgaccacaacgttccggctaacaccctgaaagctgctaacatgcagaagatcactcgcgaattcatcaaagagcagaacatcatcaaccactacctggacggtgaaggtgtttgccaccaggtactgccggaaaacggtcacattcagccgaacatggttatcgctggcggcgattctcacacctgtacttacggcgcattcggtgcgttcgctactggcttcggtgcaactgacatgggtaacatctacgcaactggtaaaacctggctgaaagttccgaaaactattcgtatcaacgttaacggtgaaaacgacaagatcaccggtaaagacatcatcctgaaaatctgcaaagaagttggtcgttctggtgcaacttacatggcgctggaatacggtggtgaagcaatcaagaaactgtctatggacgaacgtatggttctgtctaacatggctatcgaaatgggtggtaaagttggtctgatcgaagctgacgaaaccacttacaactatctgcgtaacgttggtatttctgaagagaagatcctggaactgaagaaaaaccagatcactatcgacgaaaacaacatcgacaacgacaactactacaaaatcatcaacatcgacatcactgacatggaagaacaggttgcttgcccgcaccacccggataacgttaaaaacatctctgaagttaaaggcgcaccaatcaaccaggtattcatcggttcctgcaccaacggtcgcctgaacgatctgcgcattgcttctaaatacctgaaaggtaagaaagttcacaacgacgtacgtctgatcgttatcccggcttccaagtctatcttcaagcaggcgctgaaagaaggtctgatcgacatcttcgttgacgctggcgcgctgatctgcactccgggttgcggtccgtgcctgggtgcacaccagggcgtactgggtgacggtgaagtttgcctggcaactaccaaccgtaacttcaaaggtcgtatgggtaacaccactgctgaaatctacctgtcctctccggcaatcgctgctaaatctgctatcaaaggttacatcactaacgagtaataaggtaccaggaggaattaaccatatgatcatcaaaggtaacatccacctgttcggtgacgacatcgacactgacgctatcatcccaggtgcttacctgaaaaccactgacccgaaagagctggcatctcactgcatggcgggtatcgacgaaaaattctctaccaaagttaaagacggtgacatcatcgttgctggcgaaaacttcggttgcggttcttcccgtgaacaggcaccgatctccatcaagcacaccggtatcaaagcggttgttgctgaatccttcgctcgcattttctaccgtaactgcatcaacatcggtctgatcccgatcacctgtgaaggtatcaacgaacagattcagaacctgaaagacggtgacaccatcgaaatcgatctgcagaacgaaaccatcaagatcaactccatgatgctgaactgcggtgcaccgaaaggtatcgaaaaagaaatcctggatgctggcggtctggtacagtacaccaagaacaagctgaagaaataataacccgggaagcttgagctcgaattcactggccgtcgttttacagccaagcttggctgttttggcggatgagagaagattttcagcctgatacagattaaatcagaacgcagaagcggtctgataaaacagaatttgcctggcggcagtagcgcggtggtcccacctgaccccatgccgaactcagaagtgaaacgccgtagcgccgatggtagtgtggggtctccccatgcgagagtagggaactgccaggcatcaaataaaacgaaaggctcagtcgaaagactgggcctttcgttttatctgttgtttgtcggtgaacgctctcctgagtaggacaaatccgccgggagcggatttgaacgttgcgaagcaacggcccggagggtggcgggcaggacgcccgccataaactgccaggcatcaaattaagcagaaggccatcctgacggatggcctttttgcgtttctacaaactcttttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtaattcgtaatcatgtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttggggggggggggaaagccacgttgtgtctcaaaatctctgatgttacattgcacaagataaaaatatatcatcatgaacaataaaactgtctgcttacataaacagtaatacaaggggtgttatgagccatattcaacgggaaacgtcttgctcgagatctatcgattttcgttcgtgaatacatgttataataactataactaataacgtaacgtgactggcaagagatatttttaaaacaatgaataggtttacacttactttagttttatggaaatgaaagatcatatcatatataatctagaataaaattaactaaaataattattatctagataaaaaatttagaagccaatgaaatctataaataaactaaattaagtttatttaattaacaactatggatataaaataggtactaatcaaaatagtgaggaggatatatttgaatacatacgaacaaattaataaagtgaaaaaaatacttcggaaacatttaaaaaataaccttattggtacttacatgtttggatcaggagttgagagtggactaaaaccaaatagtgatcttgactttttagtcgtcgtatctgaaccattgacagatcaaagtaaagaaatacttatacaaaaaattagacctatttcaaaaaaaataggagataaaagcaacttacgatatattgaattaacaattattattcagcaagaaatggtaccgtggaatcatcctcccaaacaagaatttatttatggagaatggttacaagagctttatgaacaaggatacattcctcagaaggaattaaattcagatttaaccataatgctttaccaagcaaaacgaaaaaataaaagaatatacggaaattatgacttagaggaattactacctgatattccattttctgatgtgagaagagccattatggattcgtcagaggaattaatagataattatcaggatgatgaaaccaactctatattaactttatgccgtatgattttaactatggacacgggtaaaatcataccaaaagatattgcgggaaatgcagtggctgaatcttctccattagaacatagggagagaattttgttagcagttcgtagttatcttggagagaatattgaatggactaatgaaaatgtaaatttaactataaactatttaaataacagattaaaaaaattataaaaaaattgaaaaaatggtggaaacacttttttcaatttttttgttttattatttaatatttgggaaatattcattctaattggtaatcagattttagaaaacaataaacccttgcatatgatatcgatgtacagatccctggtatgagtcagcaacaccttcttcacgaggcagacctcagcgcccccccccccctagcttgtctacgtctgatgctttgaatcggacggacttgccgatcttgtatgcggtgatttttccctcgtttgcccactttttaatggtggccggggtgagagctacgcgggcggcgacctgctgcgctgtgatccaatattcggggtcgttcactggttcccctttctgatttctggcatagaagaacccccgtgaactgtgtggttccgggggttgctgatttttgcgagacttctcgcgcaattccctagcttaggtgaaaacaccatgaaacactagggaaacacccatgaaacacccattagggcagtagggcggcttcttcgtctagggcttgcatttgggcggtgatctggtctttagcgtgtgaaagtgtgtcgtaggtggcgtgctcaatgcactcgaacgtcacgtcatttaccgggtcacggtgggcaaagagaactagtgggttagacattgttttcctcgttgtcggtggtggtgagcttttctagccgctcggtaaacgcggcgatcatgaactcttggaggttttcaccgttctgcatgcctgcgcgcttcatgtcctcacgtagtgccaaaggaacgcgtgcggtgaccacgacgggcttagcctttgcctgcgcttctagtgcttcgatggtggcttgtgcctgcgcttgctgcgcctgtagtgcctgttgagcttcttgtagttgctgttctagctgtgccttggttgccatgctttaagactctagtagctttcctgcgatatgtcatgcgcatgcgtagcaaacattgtcctgcaactcattcattatgtgcagtgctcctgttactagtcgtacatactcatatttacctagtctgcatgcagtgcatgcacatgcagtcatgtcgtgctaatgtgtaaaacatgtacatgcagattgctgggggtgcagggggcggagccaccctgtccatgcggggtgtggggcttgccccgccggtacagacagtgagcaccggggcacctagtcgcggataccccccctaggtatcggacacgtaaccctcccatgtcgatgcaaatctttaacattgagtacgggtaagctggcacgcatagccaagctaggcggccaccaaacaccactaaaaattaatagttcctagacaagacaaacccccgtgcgagctaccaactcatatgcacgggggccacataacccgaaggggtttcaattgacaaccatagcactagctaagacaacgggcacaacacccgcacaaactcgcactgcgcaaccccgcacaacatcgggtctaggtaacactgaaatagaagtgaacacctctaaggaaccgcaggtcaatgagggttctaaggtcactcgcgctagggcgtggcgtaggcaaaacgtcatgtacaagatcaccaatagtaaggctctggcggggtgccataggtggcgcagggacgaagctgttgcggtgtcctggtcgtctaacggtgcttcgcagtttgagggtctgcaaaactctcactctcgctgggggtcacctctggctgaattggaagtcatgggcgaacgccgcattgagctggctattgctactaagaatcacttggcggcgggtggcgcgctcatgatgtttgtgggcactgttcgacacaaccgctcacagtcatttgcgcaggttgaagcgggtattaagactgcgtactcttcgatggtgaaaacatctcagtggaagaaagaacgtgcacggtacggggtggagcacacctatagtgactatgaggtcacagactcttgggcgaacggttggcacttgcaccgcaacatgctgttgttcttggatcgtccactgtctgacgatgaactcaaggcgtttgaggattccatgttttcccgctggtctgctggtgtggttaaggccggtatggacgcgccactgcgtgagcacggggtcaaacttgatcaggtgtctacctggggtggagacgctgcgaaaatggcaacctacctcgctaagggcatgtctcaggaactgactggctccgctactaaaaccgcgtctaaggggtcgtacacgccgtttcagatgttggatatgttggccgatcaaagcgacgccggcgaggatatggacgctgttttggtggctcggtggcgtgagtatgaggttggttctaaaaacctgcgttcgtcctggtcacgtggggctaagcgtgctttgggcattgattacatagacgctgatgtacgtcgtgaaatggaagaagaactgtacaagctcgccggtctggaagcaccggaacgggtcgaatcaacccgcgttgctgttgctttggtgaagcccgatgattggaaactgattcagtctgatttcgcggttaggcagtacgttctagattgcgtggataaggctaaggacgtggccgctgcgcaacgtgtcgctaatgaggtgctggcaagtctgggtgtggattccaccccgtgcatgatcgttatggatgatgtggacttggacgcggttctgcctactcatggggacgctactaagcgtgatctgaatgcggcggtgttcgcgggtaatgagcagactattcttcgcacccactaaaagcggcataaaccccgttcgatattttgtgcgatgaatttatggtcaatgtcgcgggggcaaactatgatgggtcttgttgttgacaatggctgatttcatcaggaatggaactgtcatgctgttatgtgcctggctcctaatcaaagctggggacaatgggttgccccgttgatctgatctagttcggattggcggggcttcactgtatctgggggtggcatcgtgaatagattgcacaccgtagtgggcagtgtgcacaccatagtggccatgagcaccaccacccccagggacgccgacggcgcgaagctctgcgcctggtgcggctcggagatcaagcaatccggcgtcggccggagccgggactactgccgccgctcctgccgccagcgggcgtacgaggcccggcgccagcgcgaggcgatcgtgtccgccgtggcgtcggcagtcgctcgccgagatacgtcacgtgacgaaatgcagcagccttccattccgtcacgtgacgaaactcgggccgcaggtcagagcacggttccgcccgctccggccctgccggacccccggcatcccgcaagaggcccggcagtaccggcataaccaagcctatgcctacagcatccagggtgacggtgccgaggatgacgatgagcgcattgttagatttcatacacggtgcctgactgcgttagcaatttaactgtgataaactaccgcattaaagcttatcgatgataagctgtcaaacatggcctgtcgcttgcggtattcggaatcttgcacgccctcgctcaagccttcgtcactggtcccgccaccaaacgtttcggcgagaagcaggccattatcgccggcatggcggccgacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatatccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcaccattcgatggtgtcaacgtaaatgcatgccgcttcgccttcgcgcgcgaattgcaagctgatccgggcttatcgactgcacggtgcaccaatgcttctggcgtcaggcagccatcggaagctgtggtatggctgtgcaggtcgtaaatcactgcataattcgtgtcgctcaaggcgcactcccgttctggataatgttttttgcgccgacatcataacggttctggcaaatattctgaaatgagctgttgacaattaatcatcggctcgtataatgtgtggaattgtgagcggataacaatttcacacaggaaacagaattaaaagatatgaccatgattacgcc

SEQ ID NO: 12 - DNA, 32 bp, Artificial
  primer
aaatttggtaccgctaggaggaattaaccatg

SEQ ID NO: 13 - DNA, 33 bp, Artificial
  primer
aaatttactagtaagctgggtttacgcgacttc

SEQ ID NO: 14 - DNA, 33 bp, Artificial
  primer
aaatttactagtggctaggaggaattacatatg

SEQ ID NO: 15 - DNA, 35 bp, Artificial
  primer
aaatttaagcttattacttgttctgctccgcaaac

(19) United States
(12) Patent Application Publication
(10) Pub. No.: US 2014/0113338 A1
(43) Pub. Date: Apr. 24, 2014

(54) PREPARATION OF 5-FORMYL VALERIC ACID FROM ALPHA-KETOPIMELIC ACID

(71) Applicants: Axel Christoph Trefzer, Echt (NL); Stefanus Cornelis Hendrikus Josef Turk, Echt (NL); Jan Metske Van Der Laan, Echt (NL); Iise De Lange, Echt (NL); Denise Ilse Jacobs, Echt (NL)

(72) Inventors: Axel Christoph Trefzer, Echt (NL); Stefanus Cornelis Hendrikus Josef Turk, Echt (NL); Jan Metske Van Der Laan, Echt (NL); Iise De Lange, Echt (NL); Denise Ilse Jacobs, Echt (NL)

(73) Assignee: DSM IP ASSETS B.V., Heerlen (NL)

(21) Appl. No.: 14/009,202

(22) PCT Filed: Mar. 30, 2012
(86) PCT No.: PCT/EP2012/055852

§ 371 Date: Dec. 26, 2013

(30) Foreign Application Priority Data
Apr. 1, 2011 (EP) 11160839.4

(51) Int. Cl. C12P 13/00; C12P 7/44; C12N 9/88; C12P 7/40

(52) U.S. Cl. CPC C12P 13/001; C12P 7/40; C12P 7/44; C12N 9/88
USPC 435/121; 435/128; 435/136; 435/142; 435/232; 435/252.31; 435/252.32; 435/252.33; 435/254.2; 435/254.21; 435/254.22; 435/254.23; 435/254.3; 435/254.5; 536/23.2

(57) ABSTRACT

The invention relates to an alpha-ketopimelic acid decarboxylase enzyme that is a homologue of SEQ ID NO:2, comprising at least one mutation selected from a group of substitutions listed in the specification, to a method for preparing 5-formyl valeric acid (hereinafter also referred to as ‘5-FVA’), to a method for preparing 6-aminocaproic acid (hereinafter also referred to as ‘6-ACA’), to a method for preparing ε-caprolactam (hereinafter referred to as ‘caprolactam’) from 6-ACA, to a method for the preparation of adipic acid, to a method for preparing diaminohexane. The invention further relates to a host cell which may be used in a method according to the invention and to a polynucleotide encoding an alpha-ketopimelic acid decarboxylase enzyme.

The invention relates to an alpha-ketopimelic acid decarboxylase enzyme, to a method for preparing 5-formyl valeric acid (hereinafter also referred to as ‘5-FVA’), to a method for preparing 6-aminocaproic acid (hereinafter also referred to as ‘6-ACA’), to a method for preparing ε-caprolactam (hereinafter referred to as ‘caprolactam’) from 6-ACA, to a method for the preparation of adipic acid, to a method for preparing diaminohexane. The invention further relates to a host cell which may be used in a method according to the invention and to a polynucleotide encoding an alpha-ketopimelic acid decarboxylase enzyme.

Adipic acid (hexanedioic acid) is inter alia used for the production of polyamide. Further, esters of adipic acid may be used in plasticisers, lubricants, solvents and in a variety of polyurethane resins. Other uses of adipic acid are as food acidulants, applications in adhesives, insecticides, tanning and dyeing. Known preparation methods include the oxidation of cyclohexanol or cyclohexanone or a mixture thereof (KA oil) with nitric acid.

Diaminohexane is inter alia used for the production of polyamides such as nylon 6,6. Other uses include uses as starting material for other building blocks (e.g. hexamethylene diisocyanate) and as crosslinking agent for epoxides. A known preparation method proceeds from acrylonitrile via adiponitrile.

Caprolactam is a lactam which may be used for the production of polyamide, for instance nylon-6 or nylon-6,12 (a copolymer of caprolactam and laurolactam). Various manners of preparing caprolactam from bulk chemicals are known in the art and include the preparation of caprolactam from cyclohexanone, toluene, phenol, cyclohexanol, benzene or cyclohexane. These intermediate compounds are generally obtained from mineral oil. In view of a growing desire to prepare materials using more sustainable technology it would be desirable to provide a method wherein caprolactam is prepared from an intermediate compound that can be obtained from a biologically renewable source or at least from an intermediate compound that is converted into caprolactam using a biochemical method. Further, it would be desirable to provide a method that requires less energy than conventional chemical processes making use of bulk chemicals from petrochemical origin.

It is known to prepare caprolactam from 6-ACA, e.g. as described in U.S. Pat. No. 6,194,572. As disclosed in WO 2005/068643, 6-ACA may be prepared biochemically by converting 6-aminohex-2-enoic acid (6-AHEA) in the presence of an enzyme having α,β-enoate reductase activity. The 6-AHEA may be prepared from lysine, e.g. biochemically or by pure chemical synthesis. Although the preparation of 6-ACA via the reduction of 6-AHEA is feasible by the methods disclosed in WO 2005/068643, the inventors have found that—under the reduction reaction conditions—6-AHEA may spontaneously and substantially irreversibly cyclise to form an undesired side-product, notably β-homoproline. This cyclisation may be a bottleneck in the production of 6-ACA, and may lead to a considerable loss in yield.

WO 2009/113855 discloses new reaction pathways for the preparation of 6-ACA, namely the preparation of 6-ACA from alpha-ketopimelic acid (AKP) via the intermediate 5-FVA or via the intermediate alpha-aminopimelic acid (AAP). WO 2009/113855 also discloses biocatalysts capable of catalysing at least one of the reaction steps in the preparation of 6-ACA from AKP. Although WO 2009/113855 discloses methods that are effective in producing 6-ACA, it would be desirable to provide novel biocatalysts suitable to catalyse a reaction step in the preparation of G-ACA from AKP, in particular a biocatalyst with an improved specificity towards one of the reaction steps and/or improved activity towards one of the reaction steps. More in particular, it would be desirable to provide a novel biocatalyst which is suitable to increase the production rate of biocatalytically produced 6-ACA or an intermediate therefore.

It is an object of the invention to provide a novel biocatalyst, suitable for catalysing a reaction step in the preparation of 6-ACA from AKP.

It is in particular an object of the invention to provide a method for preparing 5-FVA.

It is a further object to provide a method for preparing a compound from 6-ACA.

It is a further object to provide a method for preparing adipic acid or diaminohexane.

It is a further object to provide a novel biocatalyst or method that would overcome one or more of the drawbacks of the above mentioned prior art.

One or more further objects which may be solved in accordance with the invention, will follow from the description below.

It has now been found possible to prepare an intermediate for 6-ACA (namely 5-FVA) biocatalytically from AKP using a specific biocatalyst.

Accordingly, the invention relates to an alpha-ketopimelic acid decarboxylase enzyme having an increased specificity towards the conversion of alpha-ketopimelic acid into 5-formylvaleric acid relative to the conversion of alpha-ketoadipic acid (AKA) into 4-formylbutyric acid (4-FBA), compared to the enzyme having alpha-ketopimelic acid decarboxylase represented by SEQ ID NO:2.

The invention further relates to a nucleic acid encoding an alpha-ketopimelic acid decarboxylase enzyme having an increased specificity towards the conversion of alpha-ketopimelic acid into 5-formylvaleric acid relative to the conversion of alpha-ketoadipic acid (AKA) into 4-formylbutyric acid (4-FBA), compared to the enzyme having alpha-ketopimelic acid decarboxylase represented by SEQ ID NO:2. To the best of the inventors' knowledge a nucleic acid or enzyme according to the invention is non-existent in nature, i.e. synthetic, in particular recombinant. In general, it is isolated from its natural source, if there is any. The nucleic acid may form part of one or more vectors.

The invention further relates to a host cell comprising a gene encoding an alpha-ketopimelic acid decarboxylase enzyme having an increased specificity towards the conversion of alpha-ketopimelic acid into 5-formylvaleric acid relative to the conversion of alpha-ketoadipic acid (AKA) into 4-formylbutyric acid (4-FBA), compared to the enzyme having alpha-ketopimelic acid decarboxylase represented by SEQ ID NO:2. Said gene is, in general, heterologous to the host cell.

The invention further relates to a method for preparing 5-formylvaleric acid, comprising decarboxylating alpha-ketopimelic acid, wherein the decarboxylation is catalysed by an alpha-ketopimelic acid decarboxylase enzyme having an increased specificity towards the conversion of alpha-ketopimelic acid into 5-formylvaleric acid relative to the conversion of alpha-ketoadipic acid (AKA) into 4-formylbutyric acid (4-FBA), compared to the enzyme having alpha-ketopimelic acid decarboxylase activity represented by SEQ ID NO:2 or a host cell comprising such alpha-ketopimelic acid decarboxylase enzyme, thereby forming the 5-formylvaleric acid.

The invention further relates to a method for preparing 6-aminocaproic acid, comprising converting 5-formylvaleric acid obtained in a method according to the invention into 6-aminocaproic acid.

The invention further relates to a method for preparing caprolactam, comprising cyclising 6-aminocaproic acid obtained in a method according to the invention, thereby obtaining caprolactam.

The invention further relates to a method for preparing adipic acid, wherein, 5-FVA obtained in accordance with the invention is converted into adipic acid.

The invention further relates to a method for preparing diaminohexane, wherein, 6-ACA obtained in accordance with the invention is converted into diaminohexane.

The specificity towards the conversion of AKP into 5-FVA relative to the conversion of AKA into 4-FBA, which can be determined on the basis of determining the ratio of activity for converting AKP into 5-FVA or to the activity for converting AKA into 4-FBA, is herein after be referred to as ‘5-FVA/4-FBA’. It is contemplated that AKA reactivity is representative for shorter 2-oxo-dicarboxylic acids. So it is contemplated that a catalyst having an increased 5-FVA/4-FBA ratio is generally also thought to have reduced specificity towards the conversion of the total of 2-oxo-dicarboxylic acids, smaller than AKP, when compared to the activity for the conversion of AKP.

The present invention is in particular based on the finding that an enzyme with an increased specificity towards the conversion of AKP into 5-FVA compared to the decarboxylation of a shorter 2-oxo-dicarboxylic acid, in particular alpha-ketoadipic acid (AKA), results in an increased production of 6-ACA and adipate in a method wherein 6-ACA is prepared from AKP. The inventors further have provided a wide variety of enzymes having alpha-ketopimelic acid decarboxylase activity with increased specificity towards the conversion of AKP into 5-FVA, compared to the wild type enzyme represented by SEQ ID NO: 2. There is no suggestion in the prior art that it would be possible to increase this specificity. It is noted that Yep et al (Bioorganic Chemistry 34 (2006) 325-336) mentions mutations in KdcA from Lactococcus Lactis, namely F381W, V461I and M538W. However these mutations were made to improve activity/specificity on pyruvic acid. Table 2 shows that mutations influence activity as well as specificity, but in general the article does not really give any teaching of how one would have to shift specificity from a relatively small substrate towards a larger substrate. In particular the prior art does not mention 2-oxo-dicarboxylic acid substrates nor it provides a solution to the problem of increasing the size of the active site to allow larger substrates to enter and at the same time preventing smaller substrates to compete with these larger substrates for being converted. Thus, there is no suggestion in this document to provide such enzyme for use in a method according to the invention. In principle the mutated enzymes of Yep may be used in a method according to the invention. In a specific embodiment though, the decarboxylase (used) in accordance with the invention or present in a host cell according to the invention is another than those mentioned in Yep et al., i.e. said enzyme having AKP-decarboxylase activity has at least one other mutation than F381W, V461I or M538W or comprises at least two mutations selected from the group of F381W, V461I and M538W.

Contrary to ordinary enzyme conversions where a single substrate is contacted with the enzyme and the product of the reaction is recovered after the conversion is completed, the synthesis of 6-ACA comprises a cascade of enzymatic reactions. In such a cascade of enzymatic reactions the initial substrate is converted by a series of consecutive enzymatic conversions catalysed each by a particular enzyme into the final desired product. Each intermediate product that is produced is substrate for the next enzyme in line. However if the enzyme that is next in line also converts intermediates further back in line, it will exhaust the production line resulting in a low yield of the desired product and an excessive production of undesired by-products. In case of fermentative production this loss of intermediates will lead to a very bad product yield based on carbon feed, e.g. glucose. In addition, the abundant production of by-products puts quite a burden on the production organism which may drastically influence biomass formation and/or robustness of the process. Further, the presence of one or more undesired by-products in a large quantity generally makes the recovery of the desired product more complicated, and may result in a reduced yield of recovered product (due to product loss during recovery) or a reduced product purity.

For resting cell conversions or in vitro multi-enzyme conversion similar problems as above will occur. It might be even more serious as it is expected that living cells are able to reuse or remove side products to a certain extent, while in vitro the side products will just accumulate. In case enzymes are functioning under physiological conditions within the cell encountering multiple potential substrates, a high specificity towards a desired product is much more important than a high enzymatic activity, as metabolism would be impossible if enzymes could not distinguish between structurally similar substrates.

In the cell under physiological conditions where the enzyme encounters multiple potential substrates, the enzyme specificity is very important as it describes which part of the enzyme's activity is actually involved in the desired conversion and which part is involved in the undesired side reactions. In particular, when multiple substrates are present, the specificity of an enzyme is usually more important than its activity because side reactions generally lead to undesired by-products which are highly inefficient in respect of the yield of a particular process. Moreover, where low activity can to some extend be compensated by overexpression of the enzyme, a lack of specificity cannot be overcome. To the contrary overexpression, likely makes the situation even worse as the formation of by-products increases in the same proportion or even in a higher proportion compared to the desired reaction.

As used herein, activity of an enzyme refers to the rate at which the enzyme converts a substrate into the corresponding product. Typically, the rate is expressed as the amount of product formed by an enzyme over a certain time period under strictly defined conditions. The observed activity depends on the type of substrate, the specific reaction conditions and the dosing of the enzyme. Enzyme activity is usually expressed in units (U) which are defined as the amount of enzyme that converts 1 micromole of substrate per minute under specified conditions. In order to determine the competition between the substrates AKP and AKA for being converted by a decarboxylase, and thereby to establish whether a decarboxylase has an increased activity ratio compared to an enzyme comprising a sequence represented by SEQ ID NO:2, the enzyme is contacted with a mixture containing the substrates AKP and AKA. The corresponding reaction rates are described by v_{AKP}/v_{AKA}={d[5FVA]/dt}/{d[4FBA]/dt}=[k_{cat}/Kₘ]_{AKp}·[AKP]/[k_{cat}/Kₘ]_{AKA}·[AKA]. Given the starting concentrations [AKP]ₒ and [AKA]ₒ the amount of products formed after a certain reaction time t will allow for calculation of the relative specificity: [K_{cat}/Kₘ]_{AKP}/[k_{cat}/Kₘ]AKA={[5FVA]ₜ/[4FBA]ₜ}*{[AKA]ₒ/[AKP]ₒ}. In case [AKA]ₒ=[AKP]ₒ the ratio [5FVA]/[4FBA] is a direct indication of how much of the undesired decarboxylation of AKA took place at the cost of the desired decarboxylation of AKP. Preferably the initial [5FVA]/[4FBA] ratio is measured as the ratio is dependent on the progress of the reaction. The initial [5FVA]/[4FBA] ratio can be determined with sufficient accuracy by carrying out the reaction until a sufficiently high conversion of AKP into 5-FVA is reached, usually at least 10%, preferably at least 20%, at least 30%, at least 40%, at least 50%, most preferably at least 60% and then constructing a graph of the [5FVA]/[4FBA] ratio versus conversion and extrapolating it to 0% conversion. It is desirable to determine the initial [5FVA]/[4FBA] ratio through extrapolation, since this improves the accuracy of the determination of the initial [5FVA]/[4FBA] ratio. For accurate determination it is desirable to have sufficient data points, for instance, at least three data points, which should preferably represent a difference in conversion of at least 5%. When for screening purposes a large number of variants has to be tested, the ratio [5FVA]/[4FBA] may be determined using only one measurement of [5FVA] and [4FBA] assuming constant rates at the initial phase of the conversion. In such a case, preferably [5FVA] and [4FBA] are measured at 25% conversion of AKP into 5-FVA or less, more preferably 20% or less, 15% or less, 10% or less, most preferably not more than 5% conversion.

In practice, the specificity towards the conversion of alpha-ketopimelic acid into 5-formylvaleric acid relative to the conversion of alpha-ketoadipic acid into 4-formylbutyric acid as used herein is a method essentially as described in Example 1 (at 30° C., pH 6.7 in an aqueous solution initially comprising equimolar amounts of 5-FVA and 4-FBA, more in particular initially comprising 25 mM AKP and 25 mM AKA), with the proviso that instead of allowing the incubation to proceed for a fixed time (16 hrs), the incubation is allowed to proceed until a predetermined conversion of AKP has been reached. This predetermined AKP into 5-FVA conversion is usually chosen in the range of 1-90%, in particular in the range of 5-50% more in particular in the range of 10-40%. Specifically, in practice the predetermined AKP into 5-FVA conversion is chosen at 10%.

It should be noted that the absolute activity (as may be expressed in terms units/ml or units/g) can be lower, the same or higher than the absolute activity of the wild type enzyme. An alpha-ketopimelic acid decarboxylase with a lower absolute activity is still considered advantageous, because of its improved substrate specificity. Further, it is envisaged that at least in a specific embodiment, the expression of the alpha-ketopimelic acid decarboxylase gene is improved compared to the expression of the wild type gene.

5-FVA/4-FBA can be determined by measuring the amount of formed 5-FVA and 4-FBA, e.g. by NMR.

The 5-FVA/4-FBA of an alpha-ketopimelic acid decarboxylase enzyme according to the invention preferably has a 5-FVA/4-FBA of 1.25 or more, preferably 1.5 or more, more preferably 2.0 or more, in particular 3.0 or more or 4.0 or more (under the conditions specified in Example 1), more in particular 10 or more. In principle the improvement may be such that no 4-FBA is detectible, resulting an infinite value for the ratio. In practice, some 4-FBA may be detectible. Accordingly, 5-FVA/4-FBA may be 1000 or less, 500 or less, 100 or less, 50 or less, 35 or less or 30 or less (in particular, under the conditions specified in Example 1 or as described above).

It is envisaged that a method of the invention allows a comparable or better 5-FVA yield than the method described in WO 2009/113855. It is envisaged that a method of the invention may in particular be favourable if use is made of a living organism—in particular in a method wherein growth and maintenance of the organism is taken into account.

It is further envisaged that in an embodiment of the invention the productivity of 5-FVA or 6-ACA (g/l.h formed) in a method of the invention is improved.

The term “or” as used herein is defined as “and/or” unless specified otherwise.

The term “a” or “an” as used herein is defined as “at least one” unless specified otherwise.

When referring to a noun (e.g. a compound, an additive, etc.) in the singular, the plural is meant to be included.

When referring herein to carboxylic acids or carboxylates, e.g. 6-ACA, another amino acid, 5-FVA or AKP, these terms are meant to include the protonated carboxylic acid group (i.e. the neutral group), their corresponding carboxylate (their conjugated bases) as well as salts thereof. When referring herein to amino acids, e.g. 6-ACA, this term is meant to include amino acids in their zwitterionic form (in which the amino group is in the protonated and the carboxylate group is in the deprotonated form), the amino acid in which the amino group is protonated and the carboxylic group is in its neutral form, and the amino acid in which the amino group is in its neutral form and the carboxylate group is in the deprotonated form, as well as salts thereof.

When referring to a compound of which stereoisomers exist, the compound may be any of such stereoisomers or a combination thereof. Thus, when referred to, e.g., an amino acid of which enantiomers exist, the amino acid may be the L-enantiomer, the D-enantiomer or a combination thereof. In case a natural stereoisomer exists, the compound is preferably a natural stereoisomer.

When an enzyme is mentioned with reference to an enzyme class (EC) between brackets, the enzyme class is a class wherein the enzyme is classified or may be classified, on the basis of the Enzyme Nomenclature provided by the Nomenclature Committee of the International Union of Biochemistry and Molecular Biology (NC-IUBMB), which nomenclature may be found at http://www.chem.qmul.ac.uk/iubmb/enzyme/. Other suitable enzymes that have not (yet) been classified in a specified class but may be classified as such, are meant to be included.

Homologues typically have an intended function in common with the polynucleotide respectively polypeptide of which it is a homologue, such as encoding the same peptide respectively being capable of catalyzing the same reaction. The term homologue is also meant to include nucleic acid sequences (polynucleotide sequences) which differ from another nucleic acid sequence due to the degeneracy of the genetic code and encode the same polypeptide sequence.

The term “homologue” is used herein in particular for polypeptides having a sequence identity of at least 50%, at least 60%, at least 70%, at least 80%, at least 90% or at least 95%.

As used herein, the term “functional analogues” of a polynucleotide at least includes other sequences encoding a polypeptide having the same amino acid sequence and other sequences encoding a homologue of such polypeptide.

In particular, preferred functional analogues are nucleotide sequences having a similar, the same or a better level of expression in a host cell of interest as the nucleotide sequence of which it is referred to as being a functional analogue of.

Amino acid or nucleotide sequences are said to be homologous when exhibiting a certain level of similarity. Two sequences being homologous indicate a common evolutionary origin. Whether two homologous sequences are closely related or more distantly related is indicated by “percent identity” or “percent similarity”, which is high or low respectively.

For the purpose of this invention, it is defined here that in order to determine the percent identity of two amino acid sequences or of two nucleic acid sequences, the complete sequences are aligned for optimal comparison purposes. In order to optimize the alignment between the two sequences gaps may be introduced in any of the two sequences that are compared. Such alignment is carried out over the full length of the sequences being compared. Alternatively, the alignment may be carried out over a shorter length, for example over about 20, about 50, about 100 or more nucleic acids/based or amino acids. The identity is the percentage of identical matches between the two sequences over the reported aligned region.

A comparison of sequences and determination of percent identity between two sequences can be accomplished using a mathematical algorithm. The skilled person will be aware of the fact that several different computer programs are available to align two sequences and determine the homology between two sequences (Kruskal, J. B. (1983) An overview of squence comparison In D. Sankoff and J. B. Kruskal, (ed.), Time warps, string edits and macromolecules: the theory and practice of sequence comparison, pp. 1-44 Addison Wesley). The percent identity between two amino acid sequences can be determined using the Needleman and Wunsch algorithm for the alignment of two sequences. (Needleman, S. B. and Wunsch, C. D. (1970) J. Mol. Biol. 48, 443-453). The algorithm aligns amino acid sequences as well as nucleotide sequences. The Needleman-Wunsch algorithm has been implemented in the computer program NEEDLE. For the purpose of this invention the NEEDLE program from the EMBOSS package was used (version 2.8.0 or higher, EMBOSS: The European Molecular Biology Open Software Suite (2000) Rice, P. Longden, I. and Bleasby, A. Trends in Genetics 16, (6) pp 276-277, http://emboss.bioinformatics.nl/). For protein sequences, EBLOSUM62 is used for the substitution matrix. For nucleotide sequences, EDNAFULL is used. Other matrices can be specified. The optional parameters used for alignment of amino acid sequences are a gap-open penalty of 10 and a gap extension penalty of 0.5. The skilled person will appreciate that all these different parameters will yield slightly different results but that the overall percentage identity of two sequences is not significantly altered when using different algorithms.

The homology or identity between the two aligned sequences is calculated as follows: Number of corresponding positions in the alignment showing an identical amino acid in both sequences divided by the total length of the alignment after subtraction of the total number of gaps in the alignment. The identity defined as herein can be obtained from NEEDLE by using the NOBRIEF option and is labelled in the output of the program as “longest-identity”. For purposes of the invention the level of identity (homology) between two sequences (amino acid or nucleotide) is calculated according to the definition of “longest-identity” as can be carried out by using the program NEEDLE.

The polypeptide sequences of the present invention can further be used as a “query sequence” to perform a search against sequence databases, for example to identify other family members or related sequences. Such searches can be performed using the BLAST programs. Software for performing BLAST analyses is publicly available through the National Center for Biotechnology Information (http://www.ncbi.nlm.nih.gov). BLASTP is used for amino acid sequences and BLASTN for nucleotide sequences. The BLAST program uses as defaults:

Cost to open gap: default=5 for nucleotides/11 for proteins

Cost to extend gap: default=2 for nucleotides/1 for proteins

Penalty for nucleotide mismatch: default=−3

Reward for nucleotide match: default=1

Expect value: default=10

Wordsize: default=11 for nucleotides/28 for megablast/3 for proteins

Furthermore the degree of local identity (homology) between the amino acid sequence query or nucleic acid sequence query and the retrieved homologous sequences is determined by the BLAST program. However only those sequence segments are compared that give a match above a certain thresshold. Accordingly the program calculates the identity only for these matching segments. Therefore the identity calculated in this way is referred to as local identity.

The term ‘biocatalyst’ is used herein for a biological material or moiety derived from a biological source (for instance an organism or a biomolecule derived there from) having catalytic activity with respect to a chemical reaction step in, particular a chemical reaction step in a method according to the invention. The biocatalyst typically comprises an alpha-ketopimelic acid decarboxylase enzyme according to the invention, or at least a gene encoding such enzyme, such that the biocatalyst produces said enzyme. The biocatalyst may be used in any form. In an embodiment, one or more enzymes are used isolated from the natural environment (isolated from the organism it has been produced in), for instance as a solution, an emulsion, a dispersion, (a suspension of) freeze-dried cells, as a lysate, or immobilised on a support. In an embodiment, one or more enzymes form part of a living organism (such as living whole cells).

The enzyme may perform a catalytic function inside the cell. It is also possible that the enzyme may be secreted into a medium, wherein the cells are present.

Living cells may be growing cells, resting or dormant cells (e.g. spores) or cells in a stationary phase. It is also possible to use an enzyme forming part of a permeabilised cell (i.e. made permeable to a substrate for the enzyme or a precursor for a substrate for the enzyme or enzymes).

A biocatalyst used in a method of the invention may in principle be any organism, or be obtained or derived from any organism. The organism may be eukaryotic or prokaryotic. In particular the organism may be selected from animals (including humans), plants, bacteria, archaea, yeasts and fungi.

It will be clear to the person skilled in the art that use can be made of a naturally occurring biocatalyst (wild type) or a mutant of a naturally occurring biocatalyst with suitable activity in a method according to the invention. Properties of a naturally occurring biocatalyst may be improved by biological techniques known to the skilled person in the art, such as e.g. molecular evolution or rational design. Mutants of wild-type biocatalysts can for example be made by modifying the encoding DNA of an organism capable of acting as a biocatalyst or capable of producing a biocatalytic moiety (such as an enzyme) using mutagenesis techniques known to the person skilled in the art (random mutagenesis, site-directed mutagenesis, directed evolution, gene recombination, etc.). In particular the DNA may be modified such that it encodes an enzyme that differs by at least one amino acid from the wild-type enzyme, so that it encodes an enzyme that comprises one or more amino acid substitutions, deletions and/or insertions compared to the wild-type, or such that the mutants combine sequences of two or more parent enzymes or by effecting the expression of the thus modified DNA in a suitable (host) cell. The latter may be achieved by methods known to the skilled person in the art such as codon optimisation or codon pair optimisation, e.g. based on a method as described in WO 2008/000632.

A mutant biocatalyst may have improved properties, for instance with respect to one or more of the following aspects: selectivity towards the substrate, specificity towards the substrate, activity, stability, robustness, solvent tolerance, pH profile, temperature profile, substrate profile, susceptibility to inhibition, cofactor utilisation and substrate-affinity. Mutants with improved properties can be identified by applying e.g. suitable high through-put screening or selection methods based on such methods known to the skilled person in the art.

When referred to a biocatalyst from a particular source, recombinant biocatalysts originating from a first organism, but actually produced in a (genetically modified) second organism, are specifically meant to be included as biocatalysts, in particular enzymes, from that first organism.

The alpha-ketopimelic acid decarboxylase enzyme of the invention may, in general, be classified under EC 4.1.1 (carboxylyases). The alpha-ketopimelic acid decarboxylase enzyme may in particular be a thiamine-diphosphate dependent enzyme (ThDP-dependent enzyme).

The present invention in particular relates to an alpha-ketopimelic acid decarboxylase enzyme which is a homologue of the alpha-ketopimelic acid decarboxylase enzyme represented by SEQ ID NO:2, said homologue comprising at least one mutation in this amino acid sequence.

The mutation may be an insertion (one or more additional amino acid units introduced between two amino acid units), an extension (one or more additional amino acid units added at the N-terminus or the C-terminus of the sequence), a deletion (an amino acid unit removed from the sequence) or a substitution (an amino acid unit of the sequence replaced by a different amino acid unit). The mutation may in particular be a mutation whereby the alpha-ketopimelic acid decarboxylase activity is increased or whereby the alpha-ketoadipic acid decarboxylase activity is decreased. Good results have been achieved with an alpha-ketopimelic acid decarboxylase enzyme comprising a single substitution compared to SEQ ID NO:2. In a specific embodiment, the number of substitutions is at least 2, at least 4, or at least 6. The number of substitutions may be e.g. be 100 or less, 25 or less, 10 or less, or 7 or less.

The alpha-ketopimelic acid decarboxylase according to the invention may also comprise either an extension or a deletion of one or more amino acid units at the C-terminus and/or either an extension or a deletion of one or more amino acid units at the N-terminus compared to SEQ ID NO:2

In a preferred embodiment, the alpha-ketopimelic acid decarboxylase according to the invention is a homologue of SEQ ID NO:2 having at least one mutation, which mutation is a mutation at an amino acid position corresponding to F72, T101, V104, V111, V166, N240, F241, N258, L261, T284, A290, F291, Q377, F381, F382, V461, I465, P532, L534, L535, M538, G539, L541, F542, Q545, N546 or K547 in SEQ ID NO:2. The mutation may in particular be a substitution.

Accordingly, the present invention in particular relates to an alpha-ketopimelic acid decarboxylase enzyme, comprising an amino acid sequence according to SEQ ID NO:4, or a homologue thereof, wherein at least one of the X's in the sequence represents an amino acid unit that is different from the corresponding amino acid unit in SEQ ID NO:2.

A further improved 5-FVA/4-FBA ratio has been observed with an alpha-ketopimelic acid decarboxylase enzyme that is a homologue of SEQ ID NO:2 and that comprises a mutation, in particular a substitution at the amino acid unit corresponding to T101, V104, V111, N240, F241, L261, A290, Q377, F381, F382, V461, I465, M538, G539, F542, Q545, N546 or K547 in SEQ ID NO:2, in particular at the amino acid unit corresponding to L261, Q377, F382, V461, M538, G539, F542, N546 or K547 in SEQ ID NO:2, more in particular at an amino acid position corresponding to L261, Q377, F382, M538, F542, N546 or K547 in SEQ ID NO:2.

In particular, good results with respect to providing an alpha-ketopimelic acid decarboxylase enzyme with increased 5-FVA/4-FBA, have been achieved by providing an alpha-ketopimelic acid decarboxylase enzyme having at least 50% sequence identity with SEQ ID NO:2, wherein the enzyme comprises at least one mutation selected from the group of substitutions corresponding to 072L, 072M, 101D, 101E, 101F, 101L, 104D, 104Q, 104W, 111M, 166K, 166R, 240A, 240G, 241L, 241N, 241R, 258R, 261A, 261D, 261G, 261W, 261Y, 284C, 284I, 284S, 284V, 290E, 290F, 290N, 290Q, 290Y, 291S, 377A, 377I, 377L, 377M, 377T, 377V, 381H, 382A, 382C, 382E, 382I, 382K, 382N, 382R, 382S, 382V, 382Y, 461I, 461L, 461M, 461T, 465C, 465F, 465L, 465M, 532C, 532T, 534G, 535A, 535C, 535G, 535Q, 535S, 538A, 538C, 538G, 538H, 538L, 538S, 538W, 539H, 539L, 539Q, 539R, 539T, 541N, 541V, 542A, 542C, 542D, 542E, 542G, 542H, 542I, 542K, 542L, 542M, 542N, 542Q, 542R, 542S, 542T, 542V, 542W, 545C, 545D, 545E, 545F, 545K, 545R, 545S, 545T, 545V, 545W, 546A, 546E, 546F, 546G, 546H, 546P, 546T, 546V, 546W, 546Y and 547P in SEQ ID NO:2, with the provisio that if the enzyme has only one mutation compared to SEQ ID NO:2, that mutation is not 461I or 538W in SEQ ID NO:2. Accordingly, in a specifically preferred embodiment, the invention relates to an alpha-ketopimelic acid decarboxylase enzyme, comprising an amino acid sequence according to SEQ ID NO:5, or a homologue thereof. Herein, each set of preferred substitutions (L or M at position 72) is indicated, wherein in at least one of the shown sets the amino acid unit is different from the corresponding amino acid unit in SEQ ID NO:2.

Particularly good results have amongst others been realised with an alpha-ketopimelic acid decarboxylase enzyme which is a homologue of SEQ ID NO:2 and which contains a substitution corresponding to 382R in SEQ ID NO:2. In a further embodiment the enzyme has a substitution of an amino acid occurring at a position corresponding to 382 in SEQ ID NO:2 with arginine.

A ‘corresponding position’ refers to the vertical column in an amino acid sequence alignment between SEQ ID NO:2 and one or more homologues corresponding to a specific position in SEQ ID NO:2 and showing all the amino acids that occur at this position in the other aligned homologues (Table 1). A “corresponding substitution” refers to a substitution of an amino acid occurring at a “corresponding position” in SEQ ID NO:2 with another amino acid.

In Table 1, SEQ ID NO:2 was used as a query to perform a BLAST search on the NCBI non-redundant databases and on the DERWENT sequence databases. In total 132 hits were observed with a sequence identity of at least 50%. After removing the redundancy only 18 unique sequences were remaining, which have the following accession codes: >AXB93603, >AXB93602, >AXB93638 >AXB93604, >AXB93648, >D2BR82_LACLK, >Q684J7_LACLL, >ATD14863, >AYL70305, >AYL70309, >AYL70405, >AYL70313, >AYL70307, >AYL70406, >AYL70311, >374673372, >Q9CG07_LACLA, >F2HLY6_LACLV. Sequences Q6QBS4_{—}9LACT and AYJ51086 refer to hits which are 100% identical to SEQ ID NO:2. The multiple alignment was generated with CLUSTALW. All amino acids which are identical to SEQ ID NO:2 are represented by a dot. Deletions are represented by “−”. “ [symbol] ” indicates the corresponding position of the mutations in the decarboxylase enzymes according to the invention.

TABLE 1
Multiple sequence alignment of alpha-ketopimelic acid decarboxylase enzymes that
are homologous of SEQ ID NO: 2.

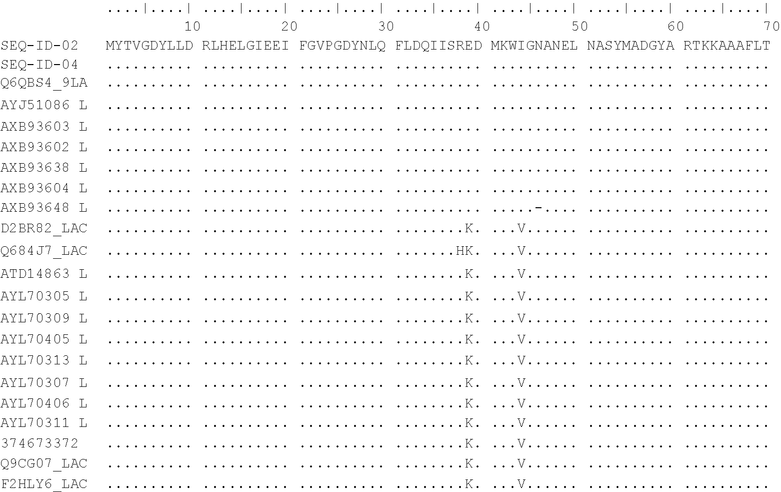

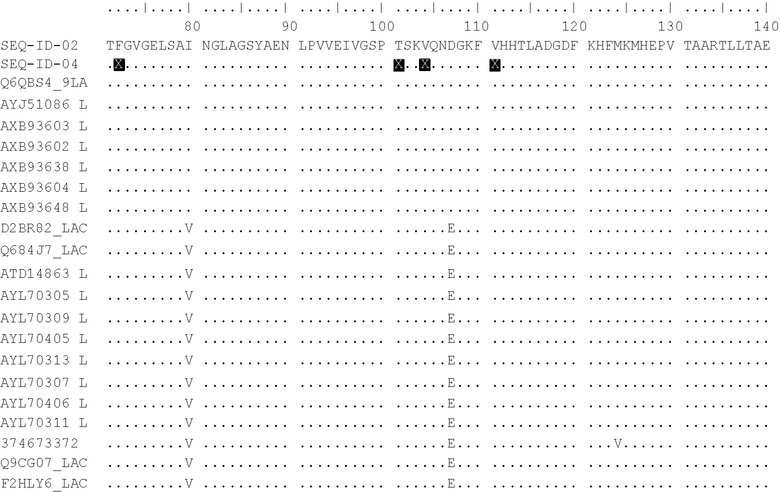

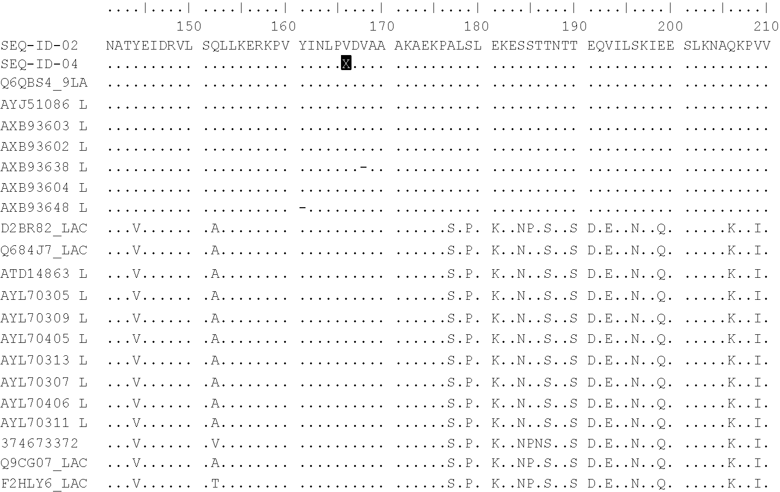

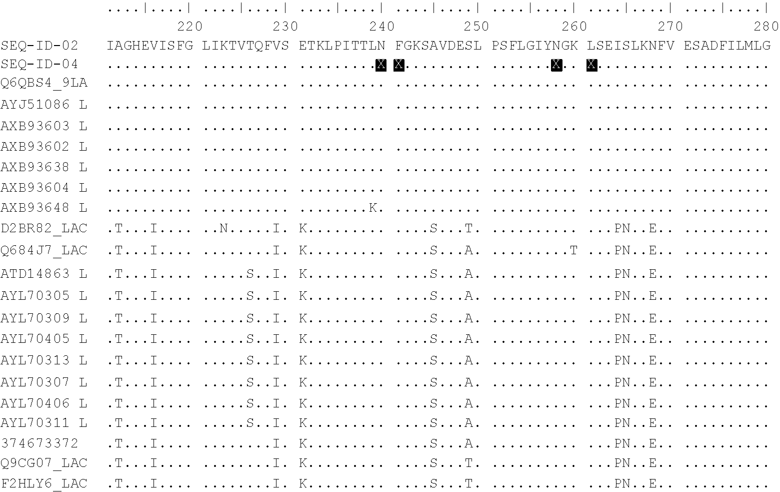

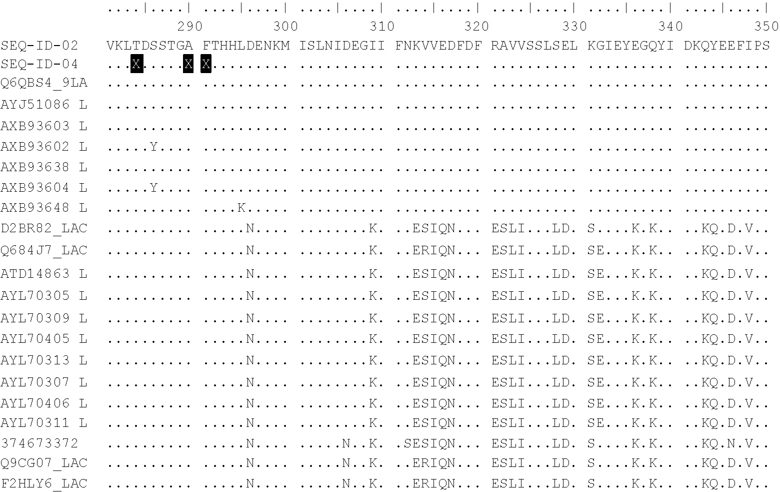

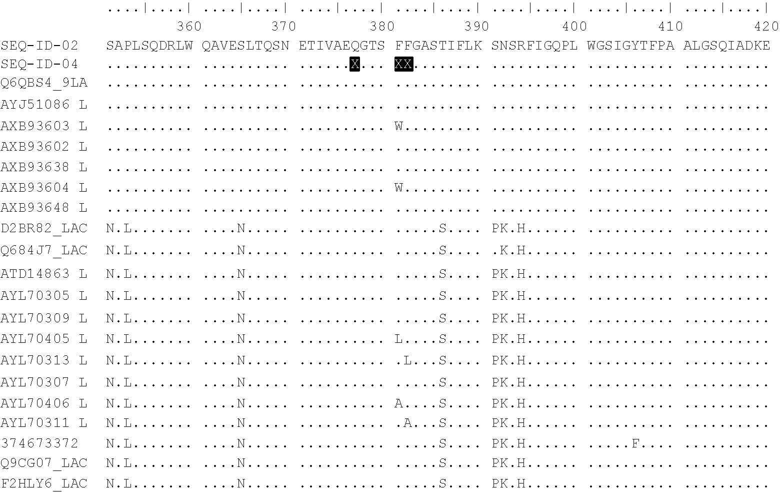

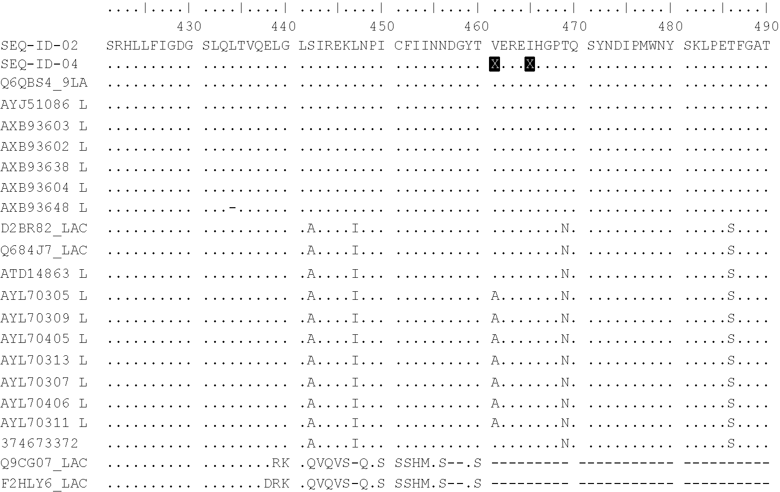

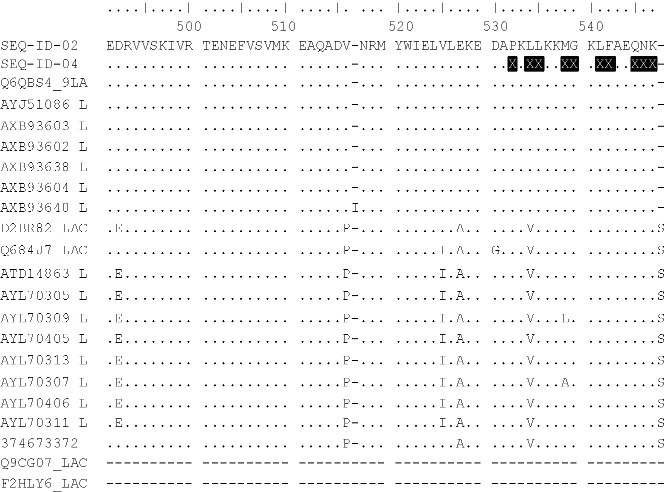

A mutation may not only affect 5-FVA/4-FBA, but also the absolute activity with respect to the decarboxylation of AKP thereby forming 5-FVA. An alpha-ketopimelic acid decarboxylase enzyme with reduced AKP decarboxylase activity yet improved specificity is still considered advantageous over the wild type enzyme represented by SEQ ID NO:2, since less side-product may be formed. In an advantageous embodiment, the AKP decarboxylase activity is about the same as or higher than the activity of said wild type enzyme (in particular under the conditions described above in general when discussing 5-FVA/4-FBA, and in detail in Example 1). Said AKP decarboxylase activity preferably is at least 0.5 times, in particular at least 1.0 times, more in particular at least 1.5 times, or at least 2.0 times the activity of the wild type enzyme represented by SEQ ID NO:2. The activity may be up to 3 times, up to 5 times, up to 10 times higher, or even higher.

An AKP decarboxylase activity that is at least about the same as the activity of the wild type enzyme represented by SEQ ID NO:2 has been observed with an alpha-ketopimelic acid decarboxylase enzyme having at least 50% sequence identity with SEQ ID NO:2 and comprising at least one mutation, in particular one substitution, at the amino acid unit corresponding to F72, T101, V111, N240, F241, L261, T284, A290, Q377, F382, V461, L534, L535, M538, G539, L541, or F542 in SEQ ID NO:2, The substitution may in particular be a substitution selected from the group of 072L, 072M, 101D, 111M, 240A, 240G, 241L, 241R, 261A, 261G, 261Y, 284I, 290F, 290N, 377I, 377L, 377M, 382A, 382C, 382E, 382R, 382Y, 461I, 461L, 461T, 534G, 535A, 535C, 535S, 538A, 538C, 539T, 541V, 542I and 542L. An increased AKP decarboxylase activity has in particular been observed for an AKP decarboxylase having at least one of the following substitutions in the amino acid position corresponding to SEQ ID NO:2: 290F, 382R, 461I, 534G, 535C.

In a specific embodiment, the AKP decarboxylase enzyme according to the invention has 50% sequence identity with SEQ ID NO:2 and comprises a sequence having two or more mutations, in particular three or more mutations, more in particular four or more mutations.

In such case, at least one of said mutations, in particular two or more of said mutations, are mutations at a position corresponding to L261, Q377, F382, M538, F542, N546 or K547 in SEQ ID NO:2. Said one or more mutations may in particular be selected from substitutions. In an embodiment comprising two or more substitutions:

L261 may in particular be substituted by G, A, Y or D;
Q377 may in particular be substituted by M, I, L, V;
F382 may in particular be substituted by E, C, N, R or S;
M538 may in particular be substituted by A, C, L, S, W or G;
F542 may in particular be substituted by I, L, M V, D, C, S, W or A;
N546 may in particular be substituted by P, T or H;
K547 may in particular be substituted by P.

Alternatively, or additionally, in an embodiment comprising at least two mutations, at least one of said mutations, in particular two or more of said mutations, are mutations at a position corresponding to F382, V461, I465, L535 or F542 in SEQ ID:2. In such embodiment comprising two or more substitutions:

F382 may in particular be substituted by R, K, Q or N;
V461 may in particular be substituted by I, L or F;
I465 may in particular be substituted by L, V, A, S or N;
L535 may in particular be substituted by V, I, f or A;
F542 may in particular be substituted by R, K, Q or N.

As mentioned above, the AKP decarboxylase enzyme may be used for the preparation of 5-FVA. The AKP decarboxylase enzyme may be used isolated from a cell or as part of the cell. Suitable conditions may be based on those described in WO 2009/113855, or in the Examples herein below.

The 5-FVA obtained in accordance with the invention preferably is used for the preparation of 6-ACA. This can be done chemically: 6-ACA can be prepared in high yield by reductive amination of 5-FVA with ammonia over a hydrogenation catalyst, for example Ni on SiO₂/Al₂O₃ support, as described for 9-aminononanoic acid (9-aminopelargonic acid) and 12-aminododecanoic acid (12-aminolauric acid) in EP-A 628 535 or DE 4 322 065.

Alternatively, 6-ACA can be obtained by hydrogenation over PtO₂ of 6-oximocaproic acid, prepared by reaction of 5-FVA and hydroxylamine. (see e.g. F. O. Ayorinde, E. Y. Nana, P. D. Nicely, A. S. Woods, E. O. Price, C. P. Nwaonicha J. Am. Oil Chem. Soc. 1997, 74, 531-538 for synthesis of the homologous 12-aminododecanoic acid).

In a particularly preferred embodiment, the preparation of 6-ACA from 5-FVA is performed biocatalytically. A method for biocatalytically preparing 6-ACA from 5-FVA may in particular be based on the methodology described in WO 2009/113855, of which the contents with respect to the preparation of 6-ACA from 5-FVA, in particular the examples and aminotransferases mentioned therein, are incorporated herein by reference.

Thus, the preparation of 6-ACA from 5-FVA can be performed biocatalytically in the presence of (i) an amino donor and (ii) an aminotransferase enzyme (E.C. 2.6.1), an amino acid dehydrogenase enzyme or another biocatalyst having catalytic activity with respect to said conversion. In a particularly preferred embodiment, the 6-ACA is formed using a biocatalyst having (reversed) 6-aminocaproic acid 6-aminotransferase activity or (reversed) 6-aminocaproic acid 6-dehydrogenase activity.

The aminotransferase enzyme may in particular be selected amongst the group of β-aminoisobutyrate:α-ketoglutarate aminotransferases, β-alanine aminotransferases, aspartate aminotransferases, 4-amino-butyrate aminotransferases (EC 2.6.1.19), L-lysine 6-aminotransferase (EC 2.6.1.36), 2-aminoadipate aminotransferases (EC 2.6.1.39), 5-aminovalerate aminotransferases (EC 2.6.1.48), 2-aminohexanoate aminotransferases (EC 2.6.1.67) and lysine:pyruvate 6-aminotransferases (EC 2.6.1.71).

In an embodiment an aminotransferase enzyme may be selected amongst the group of alanine aminotransferases (EC 2.6.1.2), leucine aminotransferases (EC 2.6.1.6), alanine-oxo-acid aminotransferases (EC 2.6.1.12), β-alanine-pyruvate aminotransferases (EC 2.6.1.18), (S)-3-amino-2-methylpropionate aminotransferases (EC 2.6.1.22), L,L-diaminopimelate aminotransferase (EC 2.6.1.83).

In a specific embodiment, the conversion of 5-FVA to 6-ACA is catalysed by a biocatalyst comprising an aminotransferase enzyme comprising an amino acid sequence, described in WO 2009/113855. Preferably, the amino donor is selected from the group of ammonia, ammonium ions, amines and amino acids. Primary amines and secondary amines are suitable amines. The amino acid may have a D- or L-configuration. Examples of amino donors are alanine, glutamate, isopropylamine, 2-aminobutane, 2-aminoheptane, phenylmethanamine, 1-phenyl-1-aminoethane, glutamine, tyrosine, phenylalanine, aspartate, β-aminoisobutyrate, β-alanine, 4-aminobutyrate, and α-aminoadipate.

In a further preferred embodiment, the method for preparing 6-ACA comprises a biocatalytic reaction in the presence of an enzyme capable of catalysing a reductive amination reaction in the presence of an ammonia source, selected from the group of oxidoreductases acting on the CH—NH₂ group of donors (EC 1.4), in particular from the group of amino acid dehydrogenases (E.C. 1.4.1). In general, a suitable amino acid dehydrogenase enzyme has 6-aminocaproic acid 6-dehydrogenase activity, catalysing the conversion of 5-FVA into 6-ACA. In particular, a suitable amino acid dehydrogenase enzyme may be selected amongst the group of diaminopimelate dehydrogenases (EC 1.4.1.16), lysine 6-dehydrogenases (EC 1.4.1.18), glutamate dehydrogenases (EC 1.4.1.3; EC 1.4.1.4), and leucine dehydrogenases (EC 1.4.1.9).

AKP used to prepare 5-FVA may in principle be obtained in any way. For instance, AKP may be obtained based on a method as described by H. Jäger et al. Chem. Ber. 1959, 92, 2492-2499. AKP can be prepared by alkylating cyclopentanone with diethyl oxalate using sodium ethoxide as a base, refluxing the resultant product in a strong acid (2 M HCl) and recovering the product, e.g. by crystallisation from toluene.

It is also possible to obtain AKP from a natural source, e.g. from methanogenic Archaea, from Asplenium septentrionale, or from Hydnocarpus anthelminthica. AKP may for instance be extracted from such organism, or a part thereof, e.g. from Hydnocarpus anthelminthica seeds. A suitable extraction method may e.g. be based on the method described in A. I. Virtanen and A. M. Berg in Acta Chemica Scandinavica 1954, 6, 1085-1086, wherein the extraction of amino acids and AKP from Asplenium, using 70% ethanol, is described.

In a specific embodiment, AKP is prepared in a method comprising converting alpha-ketoglutaric acid (AKG) into alpha-ketoadipic acid (AKA) and converting alpha-ketoadipic acid into alpha-ketopimelic acid. This reaction may be catalysed by a biocatalyst. AKG may, e.g., be prepared biocatalytically from a carbon source, such as a carbohydrate, in a manner known in the art per se.

A suitable biocatalyst for preparing AKP from AKG may in particular be selected amongst biocatalysts catalysing C₁-elongation of alpha-ketoglutaric acid into alpha-ketoadipic acid and/or C₁-elongation of alpha-ketoadipic acid into alpha-ketopimelic acid.

In a specific embodiment, the preparation of AKP is catalysed by a biocatalyst comprising

a. an AksA enzyme or an homologue thereof;

b. at least one enzyme selected from the group of AksD enzymes,

AksE enzymes, homologues of AksD enzymes and homologues of AksE enzymes; and

c. an AksF enzyme or a homologue thereof.

One or more of the AksA, AksD, AksE, AksF enzymes or homologues thereof may be found in an organism selected from the group of methanogenic archaea, preferably selected from the group of Methanococcus, Methanocaldococcus, Methanosarcina, Methanothermobacter, Methanosphaera, Methanopyrus and Methanobrevibacter.

The preparation of AKP may be based on the methodology described in WO 2009/113855, of which the contents with respect to said preparation, in particular the contents on page 18, line 3 to the end of page 19 are enclosed by reference. Further, the preparation of AKP may in particular be based on the methodology described in WO 2010/104390, of which the contents with respect to said preparation, in particular the contents on page 14, line 3 to page 22, line 9 and the examples are incorporated herein by reference.

The 6-ACA obtained in a method according to the invention can be isolated from the biocatalyst, as desired. A suitable isolation method can be based on methodology commonly known in the art.

If desired, 6-ACA obtained in accordance with the invention can be cyclised to form caprolactam, e.g. as described in U.S. Pat. No. 6,194,572.

Reaction conditions for any biocatalytic step in the context of the present invention may be chosen depending upon known conditions for the biocatalyst, in particular the enzyme, the information disclosed herein and optionally some routine experimentation.

In principle, the pH of the reaction medium used may be chosen within wide limits, as long as the biocatalyst is active under the pH conditions. Alkaline, neutral or acidic conditions may be used, depending on the biocatalyst and other factors. In case the method includes the use of a micro-organism, e.g. for expressing an enzyme catalysing a method of the invention, the pH is selected such that the micro-organism is capable of performing its intended function or functions. The pH may in particular be chosen within the range of four pH units below neutral pH and two pH units above neutral pH, i.e. between pH 3 and pH 9 in case of an essentially aqueous system at 25° C. A system is considered aqueous if water is the only solvent or the predominant solvent (>50 wt. %, in particular >90 wt. %, based on total liquids), wherein e.g. a minor amount of alcohol or another solvent (<50 wt. %, in particular <10 wt. %, based on total liquids) may be dissolved (e.g. as a carbon source) in such a concentration that micro-organisms which may be present remain active. In particular in case a yeast and/or a fungus is used, acidic conditions may be preferred, in particular the pH may be in the range of pH 3 to pH 8, based on an essentially aqueous system at 25° C. If desired, the pH may be adjusted using an acid and/or a base or buffered with a suitable combination of an acid and a base.

In principle, the incubation conditions can be chosen within wide limits as long as the biocatalyst shows sufficient activity and/or growth. This includes aerobic, micro-aerobic, oxygen limited and anaerobic conditions.

Anaerobic conditions are herein defined as conditions without any oxygen or in which substantially no oxygen is consumed by the biocatalyst, in particular a micro-organism, and usually corresponds to an oxygen consumption of less than 5 mmol/l.h, in particular to an oxygen consumption of less than 2.5 mmol/l.h, or less than 1 mmol/l.h.

Aerobic conditions are conditions in which a sufficient level of oxygen for unrestricted growth is dissolved in the medium, able to support a rate of oxygen consumption of at least 10 mmol/l.h, more preferably more than 20 mmol/l.h, even more preferably more than 50 mmol/l.h, and most preferably more than 100 mmol/l.h.

Oxygen-limited conditions are defined as conditions in which the oxygen consumption is limited by the oxygen transfer from the gas to the liquid. The lower limit for oxygen-limited conditions is determined by the upper limit for anaerobic conditions, i.e. usually at least 1 mmol/l.h, and in particular at least 2.5 mmol/l.h, or at least 5 mmol/l.h. The upper limit for oxygen-limited conditions is determined by the lower limit for aerobic conditions, i.e. less than 100 mmol/l.h, less than 50 mmol/l.h, less than 20 mmol/l.h, or less than to 10 mmol/l.h.

Whether conditions are aerobic, anaerobic or oxygen limited is dependent on the conditions under which the method is carried out, in particular by the amount and composition of ingoing gas flow, the actual mixing/mass transfer properties of the equipment used, the type of micro-organism used and the micro-organism density.

In principle, the temperature used is not critical, as long as the biocatalyst, in particular the enzyme, shows substantial activity. Generally, the temperature may be at least 0° C., in particular at least 15° C., more in particular at least 20° C. A desired maximum temperature depends upon the biocatalyst. In general such maximum temperature is known in the art, e.g. indicated in a product data sheet in case of a commercially available biocatalyst, or can be determined routinely based on common general knowledge and the information disclosed herein. The temperature is usually 90° C. or less, preferably 70° C. or less, in particular 50° C. or less, more in particular or 40° C. or less.

In particular if a biocatalytic reaction is performed outside a host organism, a reaction medium comprising an organic solvent may be used in a high concentration (e.g. more than 50%, or more than 90 wt. %), in case an enzyme is used that retains sufficient activity in such a medium.

In an advantageous method 5-FVA, and if desired 6-ACA, is prepared making use of a whole cell biotransformation of the substrate for 5-FVA (AKP or a precursor for AKP), said method comprising the use of a micro-organism in which one or more biocatalysts (usually one or more enzymes) catalysing the biotransformation are produced, such as one or more biocatalysts selected from the group of biocatalysts biocatalysts capable of catalysing the conversion of AKP to 5-FVA and biocatalysts capable of catalysing the conversion of 5-FVA to 6-ACA. In a preferred embodiment the micro-organism is capable of producing a decarboxylase and/or at least one enzyme selected from amino acid dehydrogenases and aminotransferases are produced. capable of catalysing a reaction step as described above, and a carbon source for the micro-organism.

The carbon source may in particular contain at least one compound selected from the group of monohydric alcohols, polyhydric alcohols, carboxylic acids, carbon dioxide, fatty acids, glycerides, including mixtures comprising any of said compounds. Suitable monohydric alcohols include methanol and ethanol, Suitable polyols include glycerol and carbohydrates. Suitable fatty acids or glycerides may in particular be provided in the form of an edible oil, preferably of plant origin.

In particular a carbohydrate may be used, because usually carbohydrates can be obtained in large amounts from a biologically renewable source, such as an agricultural product, preferably an agricultural waste-material. Preferably a carbohydrate is used selected from the group of glucose, fructose, sucrose, lactose, saccharose, starch, cellulose and hemi-cellulose. Particularly preferred are glucose, oligosaccharides comprising glucose and polysaccharides comprising glucose.

As indicated above, the invention further relates to a host cell. The cell, in particular a recombinant cell, comprising the AKP decarboxylase can be constructed using molecular biological techniques, which are known in the art per se. For instance, if one or more biocatalysts are to be produced in a recombinant cell (which may be a heterologous system), such techniques can be used to provide a vector (such as a recombinant vector) which comprises one or more genes encoding one or more of said biocatalysts. One or more vectors may be used, each comprising one or more of such genes. Such vector can comprise one or more regulatory elements, e.g. one or more promoters, which may be operably linked to a gene encoding an biocatalyst.

As used herein, the term “operably linked” refers to a linkage of polynucleotide elements (or coding sequences or nucleic acid sequence) in a functional relationship. A nucleic acid sequence is “operably linked” when it is placed into a functional relationship with another nucleic acid sequence. For instance, a promoter or enhancer is operably linked to a coding sequence if it affects the transcription of the coding sequence.

As used herein, the term “promoter” refers to a nucleic acid fragment that functions to control the transcription of one or more genes, located upstream with respect to the direction of transcription of the transcription initiation site of the gene, and is structurally identified by the presence of a binding site for DNA-dependent RNA polymerase, transcription initiation sites and any other DNA sequences, including, but not limited to transcription factor binding sites, repressor and activator protein binding sites, and any other sequences of nucleotides known to one of skilled in the art to act directly or indirectly to regulate the amount of transcription from the promoter. A “constitutive” promoter is a promoter that is active under most environmental and developmental conditions. An “inducible” promoter is a promoter that is active under environmental or developmental regulation. The term “homologous” when used to indicate the relation between a given (recombinant) nucleic acid or polypeptide molecule and a given host organism or host cell, is understood to mean that in nature the nucleic acid or polypeptide molecule is produced by a host cell or organisms of the same species, preferably of the same variety or strain.

The promoter that could be used to achieve the expression of the nucleic acid sequences coding for the decarboxylase according to the invention or another enzyme (in particular an aminotransferase or amino acid dehydrogenase having catalytic activity with respect to the conversion of 5-FVA into 6-ACA or an enzyme having catalytic activity with respect to the preparation of AKP from a precursor for AKP), may be native to the nucleic acid sequence coding for the enzyme to be expressed, or may be heterologous to the nucleic acid sequence (coding sequence) to which it is operably linked. Preferably, the promoter is homologous, i.e. endogenous to the host cell.

If a heterologous promoter (to the nucleic acid sequence encoding for the enzyme of interest) is used, the heterologous promoter is preferably capable of producing a higher steady state level of the transcript comprising the coding sequence (or is capable of producing more transcript molecules, i.e. mRNA molecules, per unit of time) than is the promoter that is native to the coding sequence. Suitable promoters in this context include both constitutive and inducible natural promoters as well as engineered promoters, which are well known to the person skilled in the art.

A “strong constitutive promoter” is one which causes mRNAs to be initiated at high frequency compared to a native host cell. Examples of such strong constitutive promoters in Gram-positive micro-organisms include SP01-26, SP01-15, veg, pyc (pyruvate carboxylase promoter), and amyE.

Examples of inducible promoters in Gram-positive micro-organisms include, the IPTG inducible Pspac promoter, the xylose inducible PxylA promoter.

Examples of constitutive and inducible promoters in Gram-negative microorganisms include, but are not limited to, tac, tet, trp-tet, lpp, lac, lpp-lac, laclq, T7, T5, T3, gal, trc, ara (P_{BAD}), SP6, λ-P_{R}, and λ-P_{L}.

Promoters for (filamentous) fungal cells are known in the art and can be, for example, the glucose-6-phosphate dehydrogenase gpdA promoters, protease promoters such as pepA, pepB, pepC, the glucoamylase glaA promoters, amylase amyA, amyB promoters, the catalase catR or catA promoters, glucose oxidase goxC promoter, beta-galactosidase lacA promoter, alpha-glucosidase aglA promoter, translation elongation factor tefA promoter, xylanase promoters such as xlnA, xlnB, xlnC, xlnD, cellulase promoters such as eglA, eglB, cbhA, promoters of transcriptional regulators such as areA, creA, xlnR, pacC, prtT, or another promotor, and can be found among others at the NCBI website (http://www.ncbi.nlm.nih.qov/entrez/).

The term “heterologous” when used with respect to a nucleic acid (DNA or RNA) or protein refers to a nucleic acid or protein that does not occur naturally as part of the organism, cell, genome or DNA or RNA sequence in which it is present, or that is found in a cell or location or locations in the genome or DNA or RNA sequence that differ from that in which it is found in nature. Heterologous nucleic acids or proteins are not endogenous to the cell into which it is introduced, but has been obtained from another cell or synthetically or recombinantly produced. Generally, though not necessarily, such nucleic acids encode proteins that are not normally produced by the cell in which the DNA is transcribed or expressed. Similarly exogenous RNA encodes for proteins not normally expressed in the cell in which the exogenous RNA is present. Heterologous nucleic acids and proteins may also be referred to as foreign nucleic acids or proteins. Any nucleic acid or protein that one of skill in the art would recognize as heterologous or foreign to the cell in which it is expressed is herein encompassed by the term heterologous nucleic acid or protein.

In particular, a host cell or vector according to the invention may also comprise at least one nucleic acid sequence encoding an enzyme with 5-FVA aminotransferase activity.

In such an embodiment, the nucleic acid sequence encoding an enzyme with 5-FVA aminotransferase activity may in particular comprise such an amino acid sequence mentioned in WO2009/113855. One or more of said nucleic acid sequences may form part of one or more recombinant vectors.

In a specific embodiment, the host cell comprises one or more enzymes catalysing the formation of AKP from AKG (see also above). Use may be made of an enzyme system forming part of the alpha-amino adipate pathway for lysine biosynthesis. The term ‘enzyme system’ is in particular used herein for a single enzyme or a group of enzymes whereby a specific conversion can be catalysed. Said conversion may comprise one or more chemical reactions with known or unknown intermediates e.g. the conversion of AKG into AKA or the conversion of AKA into AKP. Such system may be present inside a cell or isolated from a cell. It is known that aminotransferases often have a wide substrate range. If present, it may be desired to decrease activity of one or more such enzymes in a host cell such that activity in the conversion of AKA to alpha-aminoadipate (AAA) is reduced, whilst maintaining relevant catalytic functions for biosynthesis of other amino acids or cellular components. Also a host cell devoid of any other enzymatic activity resulting in the conversion of AKA to an undesired side product is preferred.

The host cell may for instance be selected from bacteria, yeasts or fungi. In particular the host cell may be selected from the genera selected from the group of Aspergillus, Penicillium, Saccharomyces, Kluyveromyces, Pichia, Candida, Hansenula, Bacillus, Corynebacterium, Pseudomonas, Gluconobacter, Methanococcus, Methanobacterium, Methanocaldococcus and Methanosarcina and Escherichia. Herein, usually one or more encoding nucleic acid sequences as mentioned above have been cloned and expressed.

In particular, the host strain and, thus, a host cell suitable for the biochemical synthesis of 5-FVA, and if desired 6-ACA, may be selected from the group of Escherichia coli, Bacillus subtilis, Bacillus amyloliquefaciens, Corynebacterium glutamicum, Aspergillus niger, Penicillium chrysogenum, Saccharomyces cervisiae, Hansenula polymorpha, Candida albicans, Kluyveromyces lactis, Pichia stipitis, Pichia pastoris, Methanobacterium thermoautothrophicum ΔH, Methanococcus maripaludis, Methanococcus voltae, Methanosarcina acetivorans, Methanosarcina barkeri and Methanosarcina mazei host cells. In a preferred embodiment, the host cell is capable of producing lysine (as a precursor).

The host cell may be in principle a naturally occurring organism or may be an engineered organism. Such an organism can be engineered using a mutation screening or metabolic engineering strategies known in the art. In a specific embodiment, the host cell naturally comprises (or is capable of producing) one or more of the enzymes suitable for catalysing a reaction step in a method of the invention, such as one or more activities selected from the group of decarboxylases, aminotransferases and amino acid dehydrogenases capable of catalysing a reaction step in a method of the invention. For instance E. coli may naturally be capable of producing an enzyme catalysing a transamination in a method of the invention. It is also possible to provide a recombinant host cell with both a recombinant gene encoding an aminotransferase or amino acid dehydrogenase capable of catalysing a reaction step in a method of the invention and a recombinant gene encoding a decarboxylase gene capable of catalysing a reaction step in a method of the invention.

For instance a host cell may be selected of the genus Corynebacterium, in particular C. glutamicum, enteric bacteria, in particular Escherichia coli, Bacillus, in particular B. subtilis and B. methanolicus, and Saccharomyces, in particular S. cerevisiae. Particularly suitable are C. glutamicum or B. methanolicus strains which have been developed for the industrial production of lysine.

As indicated above, 5-FVA obtained in accordance with the invention may be used for the preparation of adipic acid. This can be accomplished in a manner known per se. In particular, the aldehyde group of 5-FVA may be subjected to an oxidation reaction, thereby yielding adipic acid. This may be accomplished chemically, e.g. by selective chemical oxidation, optionally including protection of the carboxylic acid group, or biocatalytically.

In a specific method of the invention, the preparation comprises a biocatalytic reaction in the presence of a biocatalyst capable of catalysing the oxidation of the aldhehyde group. The biocatalyst may use NAD⁺or NADP⁺as electron acceptor. Aldehyde dehydrogenases are biocatalysts (enzymes) catalysing an oxidation of an aldehyde group. Thus a aldehyde dehydrogenase may be used, which preferably is selective towards the substrate 5-FVA.

The enzyme for catalysing the formation of adipic acid from 5-FVA may in particular be selected from the group of oxidoreductases (EC 1.2.1), preferably from the group of aldehyde dehydrogenase (EC 1.2.1.3, EC 1.2.1.4 and EC 1.2.1.5), malonate-semialdehyde dehydrogenase (EC 1.2.1.15), succinate-semialdehyde dehydrogenase (EC 1.2.1.16 and EC 1.2.1.24); glutarate-semialdehyde dehydrogenase (EC 1.2.1.20), aminoadipate semialdehyde dehydrogenase (EC 1.2.1.31), adipate semialdehyde dehydrogenase (EC 1.2.1.63), which may also be referred to as 6-oxohexanoate dehydrogenase. Adipate semialdehyde dehydrogenase activity has been described, for example, in the caprolactam degradation pathway in the KEGG database. In particular a 6-oxohexanoate dehydrogenase may be used. An aldehyde dehydrogenase may in principle be obtained or derived from any organism. The organism may be prokaryotic or eukaryotic. In particular the organism can be selected from bacteria, archaea, yeasts, fungi, protists, plants and animals (including human).

In an embodiment the bacterium is selected from the group of Acinetobacter (in particular Acinetobacter sp. NCIMB9871), Ralstonia, Bordetella, Burkholderia, Methylobacterium, Xanthobacter, Sinorhizobium, Rhizobium, Nitrobacter, Brucella (in particular B. melitensis), Pseudomonas, Agrobacterium (in particular Agrobacterium tumefaciens), Bacillus, Listeria, Alcaligenes, Corynebacterium, and Flavobacterium.

In an embodiment the organism is selected from the group of yeasts and fungi, in particular from the group of Aspergillus (in particular A. niger and A. nidulans) and Penicillium (in particular P. chrysogenum)

In an embodiment, the organism is a plant, in particular Arabidopsis, more in particular A. thaliana.

As mentioned above, the invention relates to the preparation of 1-6 diaminohexane. The 1,6-diaminohexane may be prepared from adipic acid obtained in accordance with the invention or from 6-ACA prepared in accordance with the invention. Such conversion may be carried out based on methodology known per se.

In particular, this may be accomplished by reducing the acid groups of adipic acid or the acid group of 6-ACA. The thus formed aldehyde group(s) are thereafter transaminated. By transamination of an aldehyde group r an aminogroup is provided. Thus, adipic acid or 6-ACA can be converted into diaminohexane. This may be accomplished chemically or biocatalytically.

In a preferred method of the invention, the preparation comprises a biocatalytic reaction in the presence of a biocatalyst capable of catalysing the reduction of the acid to form an aldehyde group and/or a biocatalytic reaction in the presence of a biocatalyst capable of catalysing said transamination, in the presence of an amino donor. A method for the preparation of diaminohexane from 6-ACA may in particular be based on WO 2010/104390, of which the contents are incorporated herein by reference.

Next, the invention will be illustrated by the following examples.

### EXAMPLE 1

### Substrate Specificity Test

Growing the Strains

96-well Half-deep-well plates (VVestburg/Thermo) containing 500 μl/well 2* TY medium (16 gr/l tryptone, 10 gr/l yeast extract, 5 gr/l NaCl) with 100 ug/ml ampicillin were inoculated with the micro-organism comprising/expressing AKP decarboxylase’ (e.g. E. coli expressing (variants of) kdcA decarboxylase) and covered with a breath seal (greiner bio-one GmbH). These plates were placed in a Multitron incubator (Infors HT, bottmingen, Switzerland) and grown for 16 hours at 30° C. at 550 rpm and 80% humidity. Subsequently, 50 μl of the overnight culture was inoculated in a 2.5 ml 96-well deepwell plate (VWR) containing 950 μl/well 2*TY medium with 100 ug/ml Ampicillin and 0.02% arabinose. The plates were covered with a breathseal and incubated for 7 hours at 30° C. at 550 rpm and 80% humidity in a Multitron incubator. The cells were collected by centrifugation of the 96-well Deepwell plates for 30 minutes at 2750 rpm at 4° C. in a Multifuge 4Kr centrifuge (Heraeus, Buckinghamshire, England.). The supernatants was discarded and the cell pellets stored at −20° C. for 16 hours.

Lysis Protocol

The cellpellets were defrosted on ice and new lysis buffer was made. This lysis buffer (contained per 700 ml:35 ml phosphate buffer 1M pH 7.5; 658 ml water; 7 ml Halt Protease inhibitor cocktail (Thermo Fisher Scientific Inc. Rockford, Ill. 61105 USA); 0.861 gr MgSO₄;1,078 gr dithiothreitol (DTT); 70 mg DNAse 1 grade II; 1.4 gr Lysozym) was preheated at 25° C. and 400 μl was added to each defrosted cellpellet. Subsequently this was mixed well and the plates were covered with a deepwell cover (Thermo 96 cap sealing mats, model AB-0675). The plates were incubated for 30 min at 25° C. and in a Multitron incubator while shaking at 550 rpm. Subsequently the cell debris was spun down for 30 min at 2750 rpm in a Multifuge 4Kr centrifuge.

In Vitro Decarboxylase Assay

90 μl cell lysate containing the KdcA decarboxylase was transferred to two new 96-well deepwell plates and 510 μl reaction mixture (300 μl phosphate buffer 200 mM pH 6.5, 3 μl 1M MgCl₂, 57 μl water, 75 μl 200 mM, alpha ketoadipate (Syncom BV, Groningen, The Netherlands), 75 μl 200 mM alpha ketopimelate (Syncom BV, Groningen, The Netherlands), 0.276 mg Thiamin pyrophosphate (preheated at 25° C.) was added. The plates were covered with a deepwell cover and incubated at 25° C. in an incubator. The reaction was stopped after 16 hours of incubation by heating the reaction for 30 min at 70° C. Subsequently the reaction mixture was incubated on ice and centrifuged for 10 minutes at 2750 rpm. 450 μl of the supernatant was transferred to a new 96-well deepwell plate and 90 μl/well 20% maleic acid was added as internal standard for the NMR analysis.

It is observed that, generally, the enzyme dosage and the activity of each mutant is at forehand unknown and therefore after 16 hours the 5-FVA concentration and as a consequence the conversion will differ, depending on the dosage and activity. It is also possible that the FVA/FBA ratio is dependant on the conversion, in that it will gradually decrease as conversion increases. Therefore, it is preferred that when comparing FVA/FBA for two enzymes, FVA/FBA is compared at the same conversion of AKP, which conversion is preferably less than 100%. Most preferably the initial FVA/FBA ratio is determined.

In order to establish whether a mutant is improved in respect of its FVA/FBA ratio one could also determine for KdcA wild type the FVA/FBA ratio as a function of the conversion of AKP. On the one hand it would allow for the determination of the initial FVA/FBA ratio, on the other hand the mutant can be compared with wild type at its observed conversion.

To determine the FVA/FBA ratio as a function of the conversion of AKP, the assay as described in this example can be carried out with different dosages of the cell lysate in such a way that the AKP conversion range of 1-85% is covered by at least 5, preferably at least 10, more preferably 15 or more measurements.

NMR Analysis.

Analysis was performed using flow-NMR. The ¹H NMR spectra were recorded on a Bruker AVANCE II BEST NMR system operating at proton frequency 500 MHz and probe temperature 27° C. Spectra were recorded with; d1=1.2 seconds, PI9=52 dB, pulse program=noesygppr1d.comp. The total conversion was followed by the ratio of peak at 9.67 ppm corresponding to the aldehyde proton of both 5-FVA and 4-FBA and the internal standard peak at 6.1 ppm (maleic acid). The conversion specificity was followed by the ratio between the peak at 1.36 ppm corresponding to the proton at position 3 of 5-FVA and the internal standard peak at 6.1 ppm (maleic acid). The peak assignment for the different compounds was confirmed by the overlay with ¹H spectra of AKA, AKP, 5-FVA and 4-FBA recorded using the same condition.

### EXAMPLE 2

### Construction of a KdcA Mutant Library and In Vitro Testing Of the Substrate Specificity

To introduce the mutations in the KdcA protein (SEQ ID NO:2), the corresponding gene was optimized for expression in E. coli (SEQ ID NO:3) and cloned into the pBAD/Myc-His-DEST expression vector using the Gateway technology (Invitrogen) via the introduced attB sites and pDONR201 (Invitrogen) as entry vector as described in the manufacturer's protocols (www.invitrogen.com). This way the expression vector pBAD-kdcA was obtained (SEQ ID NO:6). For all 58 identified positions of the KdcA protein mutations were introduced by GeneArt® (Regensburg, Germany) into the pBAD-kdcA vector using their ITERATE SeqPer A16® technology. All mutations were verified by sequencing and each mutant contained a single substitution compared to the wild type decarboxylase represented by SEQ ID NO:2. The corresponding expression strains were obtained by transformation of chemically competent E. coli TOP10 (Invitrogen) with the respective pBAD-expression vectors. The strains were delivered as individual glycerol stocks in 96 wells Micro titer plates.

Using the protocol of Example 1, these KdcA mutant decarboxylases were tested. These mutants, each containing a single substitution compared to the wild type decarboxylase represented by SEQ ID NO:2, were compared with said wild type decarboxylase. The results are shown in the following table (Table 2). The codon substituted in each mutant as compared to the wild-type sequence (SEQ ID NO:2) is also shown.

The conversion % refers to the fraction of the substrate AKP that has reacted to 5-FVA. As the starting concentration of AKP is 25 mM and each molecule of AKP will react to 1 molecule 5-FVA, the conversion is calculated as 100%[5-FVA]₁₆ₕₒᵤᵣₛ/[AKP]ₒ where [AKP]ₒ refers to the start concentration of AKP which is 25 mM.

TABLE 2
Results obtained with KdcA mutants that
contain a single mutation compared to the wild
type decarboxylase represented by SEQ ID NO: 2

| | | ratio | 5-FVA (mM) | AKP |
|---|---|---|---|---|
| | | 5-FVA/4-FBA | after | conversion |
| amino | | after | 16 hours | (%) after |
| acid | codon | 16 hours | conversion | 16 hours |
| wt | — | 1.15 | 10.02 | 40.10 |
| 072L | CTG | 1.83 | 5.30 | 21.20 |
| 072M | ATG | 1.55 | 6.80 | 27.20 |
| 101C | TGT | 1.28 | 7.80 | 31.20 |
| 101D | GAT | 2.77 | 6.10 | 24.40 |
| 101E | GAA | 2.00 | 1.80 | 7.20 |
| 101F | TTT | 2.80 | 1.40 | 5.60 |
| 101I | ATT | 1.35 | 3.50 | 14.00 |
| 101K | AAA | 1.33 | 4.00 | 16.00 |
| 101L | CTG | 1.58 | 3.00 | 12.00 |
| 101P | CCG | 1.32 | 3.70 | 14.80 |
| 101Q | CAG | 1.30 | 2.60 | 10.40 |
| 101R | CGT | 1.47 | 2.20 | 8.80 |
| 103D | GAT | 1.29 | 5.30 | 21.20 |
| 104D | GAT | 2.71 | 1.90 | 7.60 |
| 104F | TTT | 1.50 | 2.10 | 8.40 |
| 104N | AAT | 1.28 | 2.30 | 9.20 |
| 104Q | CAG | 2.67 | 0.80 | 3.20 |
| 104T | ACC | 1.30 | 3.00 | 12.00 |
| 104W | TGG | 1.80 | 0.90 | 3.60 |
| 104Y | TAT | 1.33 | 1.20 | 4.80 |
| 110L | CTG | 1.40 | 5.90 | 23.60 |
| 110R | CGT | 1.29 | 2.70 | 10.80 |
| 111M | ATG | 2.20 | 7.70 | 30.80 |
| 114S | AGC | 1.39 | 7.10 | 28.40 |
| 123C | TGT | 1.33 | 0.80 | 3.20 |
| 166E | GAA | 1.35 | 3.10 | 12.40 |
| 166K | AAA | 2.00 | 1.20 | 4.80 |
| 166M | ATG | 1.34 | 3.90 | 15.60 |
| 166Q | CAG | 1.39 | 3.90 | 15.60 |
| 166R | CGT | 1.67 | 2.50 | 10.00 |
| 166W | TGG | 1.31 | 1.70 | 6.80 |
| 239P | CCG | 1.36 | 6.80 | 27.20 |
| 240A | GCA | 1.58 | 9.00 | 36.00 |
| 240G | GGT | 2.13 | 1.70 | 6.80 |
| 240S | AGC | 1.27 | 3.80 | 15.20 |
| 240V | GTT | 1.50 | 2.10 | 8.40 |
| 241L | CTG | 1.52 | 6.40 | 25.60 |
| 241N | AAT | 1.96 | 4.90 | 19.60 |
| 241R | CGT | 2.49 | 10.20 | 40.80 |
| 258G | GGT | 1.28 | 6.00 | 24.00 |
| 258R | CGT | 1.53 | 2.30 | 9.20 |
| 260C | TGT | 1.30 | 10.00 | 40.00 |
| 261A | GCA | 2.00 | 6.40 | 25.60 |
| 261D | GAT | 2.12 | 3.60 | 14.40 |
| 261G | GGT | 6.42 | 7.70 | 30.80 |
| 261H | CAT | 1.41 | 2.40 | 9.60 |
| 261M | ATG | 1.41 | 5.50 | 22.00 |
| 261W | TGG | 1.65 | 2.80 | 11.20 |
| 261Y | TAT | 2.64 | 10.30 | 41.20 |
| 284C | TGT | 1.95 | 3.70 | 14.80 |
| 284I | ATT | 1.78 | 10.30 | 41.20 |
| 284S | AGC | 1.80 | 0.90 | 3.60 |
| 284V | GTT | 1.96 | 4.70 | 18.80 |
| 290D | GAT | 1.33 | 5.70 | 22.80 |
| 290E | GAA | 1.56 | 2.50 | 10.00 |
| 290F | TTT | 2.18 | 16.60 | 66.40 |
| 290H | CAT | 1.43 | 5.30 | 21.20 |
| 290N | AAT | 1.62 | 6.30 | 25.20 |
| 290Q | CAG | 2.29 | 3.90 | 15.60 |
| 290T | ACC | 1.35 | 4.20 | 16.80 |
| 290V | GTT | 1.42 | 10.90 | 43.60 |
| 290Y | TAT | 2.58 | 3.10 | 12.40 |
| 291S | AGC | 1.60 | 0.80 | 3.20 |
| 292G | GGT | 1.33 | 0.80 | 3.20 |
| 377A | GCA | 2.29 | 1.60 | 6.40 |
| 377I | ATT | 10.71 | 7.50 | 30.00 |
| 377L | CTG | 30.50 | 6.10 | 24.40 |
| 377M | ATG | 1.79 | 6.10 | 24.40 |
| 377T | ACC | 1.63 | 2.60 | 10.40 |
| 377V | GTT | 6.80 | 3.40 | 13.60 |
| 381H | CAT | 2.92 | 3.80 | 15.20 |
| 382A | GCA | 2.59 | 7.50 | 30.00 |
| 382C | TGT | 3.24 | 11.00 | 44.00 |
| 382E | GAA | 3.79 | 9.10 | 36.40 |
| 382I | ATT | 2.33 | 2.80 | 11.20 |
| 382K | AAA | 2.25 | 0.90 | 3.60 |
| 382Q | CAG | 1.31 | 2.10 | 8.40 |
| 382R | CGT | 16.92 | 20.30 | 81.20 |
| 382S | AGT | 3.43 | 4.80 | 19.20 |
| 382V | GTT | 2.53 | 4.30 | 17.20 |
| 382Y | TAT | 2.00 | 9.40 | 37.60 |
| 461I | ATT | 2.45 | 16.20 | 64.80 |
| 461L | CTG | 3.07 | 8.30 | 33.20 |
| 461M | ATG | 2.86 | 2.00 | 8.00 |
| 461S | AGC | 1.30 | 1.30 | 5.20 |
| 461T | ACC | 1.91 | 8.40 | 33.60 |
| 464A | GCA | 1.33 | 8.50 | 34.00 |
| 464F | TTT | 1.42 | 11.50 | 46.00 |
| 464K | AAA | 1.24 | 6.30 | 25.20 |
| 464S | AGC | 1.26 | 6.80 | 27.20 |
| 464W | TGG | 1.40 | 11.60 | 46.40 |
| 465C | TGT | 1.75 | 1.40 | 5.60 |
| 465F | TTT | 2.20 | 1.10 | 4.40 |
| 465L | CTG | 2.13 | 3.20 | 12.80 |
| 465M | ATG | 1.64 | 1.80 | 7.20 |
| 465V | GTT | 1.43 | 1.00 | 4.00 |
| 468L | CTG | 1.19 | 2.50 | 10.00 |
| 475V | GTT | 1.50 | 1.20 | 4.80 |
| 532C | TGT | 1.75 | 0.70 | 2.80 |
| 532T | ACC | 1.67 | 1.00 | 4.00 |
| 534A | GCA | 1.22 | 6.10 | 24.40 |
| 534C | TGT | 1.39 | 5.00 | 20.00 |
| 534D | GAT | 1.26 | 5.90 | 23.60 |
| 534G | GGT | 1.86 | 11.90 | 47.60 |
| 534K | AAA | 1.47 | 6.30 | 25.20 |
| 534N | AAT | 1.45 | 6.10 | 24.40 |
| 534P | CCG | 1.33 | 2.00 | 8.00 |
| 534Q | CAG | 1.25 | 4.00 | 16.00 |
| 534R | CGT | 1.19 | 3.20 | 12.80 |
| 534S | AGC | 1.47 | 5.60 | 22.40 |
| 534T | ACC | 1.48 | 7.10 | 28.40 |
| 534W | TGG | 1.24 | 3.10 | 12.40 |
| 534Y | TAT | 1.42 | 1.70 | 6.80 |
| 535A | GCA | 1.67 | 10.20 | 40.80 |
| 535C | TGT | 1.84 | 12.50 | 50.00 |
| 535G | GGT | 1.75 | 3.50 | 14.00 |
| 535Q | CAG | 2.00 | 1.80 | 7.20 |
| 535S | AGC | 1.65 | 5.60 | 22.40 |
| 535T | ACC | 1.44 | 3.90 | 15.60 |
| 538A | GCA | 4.00 | 7.60 | 30.40 |
| 538C | TGT | 2.19 | 6.80 | 27.20 |
| 538G | GGT | 5.57 | 3.90 | 15.60 |
| 538H | CAT | 1.57 | 1.10 | 4.40 |
| 538Q | CAG | 1.43 | 2.00 | 8.00 |
| 538S | AGC | 3.80 | 1.90 | 7.60 |
| 538W | TGG | 3.00 | 2.70 | 10.80 |
| 538Y | TAT | 1.39 | 4.30 | 17.20 |
| 539C | TGT | 1.38 | 10.20 | 40.80 |
| 539H | CAT | 3.00 | 0.90 | 3.60 |
| 539K | AAA | 1.36 | 1.50 | 6.00 |
| 539L | CTG | 3.00 | 3.00 | 12.00 |
| 539M | ATG | 1.42 | 4.70 | 18.80 |
| 539Q | CAG | 2.14 | 1.50 | 6.00 |
| 539R | CGT | 1.78 | 1.60 | 6.40 |
| 539T | ACC | 1.68 | 5.20 | 20.80 |
| 541D | GAT | 1.38 | 4.40 | 17.60 |
| 541N | AAT | 1.75 | 0.70 | 2.80 |
| 541T | ACC | 1.40 | 2.80 | 11.20 |
| 541V | GTT | 1.82 | 6.20 | 24.80 |
| 542A | GCA | 3.09 | 3.40 | 13.60 |
| 542C | TGT | 4.43 | 3.10 | 12.40 |
| 542D | GAT | 5.67 | 3.40 | 13.60 |
| 542E | GAA | 3.20 | 1.60 | 6.40 |
| 542G | GGT | 3.00 | 1.50 | 6.00 |
| 542H | CAT | 2.13 | 1.70 | 6.80 |
| 542I | ATT | 3.38 | 9.80 | 39.20 |
| 542K | AAA | 1.60 | 0.80 | 3.20 |
| 542L | CTG | 1.58 | 9.00 | 36.00 |
| 542N | AAT | 3.25 | 1.30 | 5.20 |
| 542Q | CAG | 2.29 | 1.60 | 6.40 |
| 542R | CGT | 2.40 | 1.20 | 4.80 |
| 542S | AGC | 4.17 | 2.50 | 10.00 |
| 542T | ACC | 2.75 | 2.20 | 8.80 |
| 542V | GTT | 3.00 | 4.20 | 16.80 |
| 543H | CAT | 1.32 | 4.50 | 18.00 |
| 543I | ATT | 1.50 | 3.60 | 14.40 |
| 543L | CTG | 1.41 | 5.80 | 23.20 |
| 544W | TGG | 1.36 | 7.20 | 28.80 |
| 545C | TGT | 1.64 | 3.60 | 14.40 |
| 545D | GAT | 2.24 | 3.80 | 15.20 |
| 545E | GAA | 2.11 | 4.00 | 16.00 |
| 545F | TTT | 1.58 | 3.80 | 15.20 |
| 545G | GGT | 1.48 | 4.60 | 18.40 |
| 545H | CAT | 1.45 | 1.60 | 6.40 |
| 545I | ATT | 1.38 | 5.10 | 20.40 |
| 545K | AAA | 2.57 | 3.60 | 14.40 |
| 545N | AAT | 1.40 | 3.50 | 14.00 |
| 545R | CGT | 1.75 | 2.10 | 8.40 |
| 545S | AGC | 1.71 | 2.90 | 11.60 |
| 545T | ACC | 1.68 | 4.70 | 18.80 |
| 545V | GTT | 1.78 | 3.20 | 12.80 |
| 545W | TGG | 1.80 | 1.80 | 7.20 |
| 546A | GCA | 2.07 | 3.10 | 12.40 |
| 546E | GAA | 2.60 | 1.30 | 5.20 |
| 546F | TTT | 2.50 | 2.50 | 10.00 |
| 546G | GGT | 1.61 | 2.90 | 11.60 |
| 546H | CAT | 1.43 | 1.00 | 4.00 |
| 546P | CCG | 3.67 | 2.20 | 8.80 |
| 546Q | CAG | 1.35 | 2.30 | 9.20 |
| 546R | CGT | 1.33 | 0.80 | 3.20 |
| 546S | AGC | 1.33 | 2.80 | 11.20 |
| 546T | ACC | 1.45 | 1.60 | 6.40 |
| 546V | GTT | 2.20 | 1.10 | 4.40 |
| 546W | TGG | 1.88 | 1.50 | 6.00 |
| 546Y | TAT | 1.88 | 4.50 | 18.00 |
| 547P | CCG | 3.60 | 1.80 | 7.20 |
| 547W | TGG | 1.24 | 6.30 | 25.20 |

### EXAMPLE 3

### Testing KdcA Variants In Vivo for Improved 6-ACA Production in E. coli

Cloning of the Genes

Protein sequences for the Methanococcus aeolicus Nankai 3 homoaconitase small subunit (AksE, Maeo_{—}0652 [SEQ ID NO:204 in WO 2010/104390, Protein ID YP_{—}001324848]), homoaconitase large subunit (AksD, Maeo_{—}0311, [SEQ ID NO:192 in WO 2010/104390, Protein ID YP_{—}001324511]), the Methanococcus maripaludis S2 homoisocitrate dehydrogenase (AksF, SEQ ID NO:36 in WO 2010/104390, Protein ID NP988000), the A. vinelandii homocitrate synthase (NifV, [SEQ ID NO:75 in WO 2010/104390, Protein ID P05342]), the aminotransferase protein from Vibrio fluvialis JS17 (SEQ ID NO:2 in WO 2010/104390) and the Lactococcus lactis branched chain alpha-keto acid decarboxylase KdcA (SEQ ID NO: 2) were retrieved from databases.

All genes, except for the A. vinelandii homocitrate synthase nifV (SEQ ID NO:149 in WO 2010/104390, M17349, Beynon, J., A. Ally, M. Cannon, F. Cannon, M. Jacobson, V. Cash and D. Dean. 1987. Comparative organization of nitrogen fixation-specific genes from Azotobacter vinelandii and Klebsiella pneumoniae: DNA sequence of the nifUSV genes. J. Bacteriol. 169(9):4024-9), were optimized for E. coli and the constructs were made synthetically (Geneart, Regensburg, Germany). In the optimization procedure internal restriction sites were avoided and common restriction sites were introduced at the start and stop to allow subcloning in expression vectors. The codon optimised aminotransferase gene from Vibrio fluvialis JS17 (SEQ ID NO:3 in WO 2010/104390) was PCR amplified using Phusion DNA polymerase according to the manufacturers specifications using primer pairs AT-Vfl_for_Ec (AAATTT GGTACC GCTAGGAGGAATTAACCATG)+AT-Vfl_rev_Ec (AAATTT ACTAGT AAGCTGGGTTTACGCGACTTC). The codon optimized sequence for decarboxylase KdcA (SEQ ID NO:3) and mutant decarboxylase KdcA mutants, KdcA(F382R), KdcA (Q3771) and KdcA(Q377L) (See Example 2 for the codon changes in the sequences for KdcA mutants) were amplified using Phusion DNA polymerase according to the manufacturers specifications and using primers Kdc_for_Ec (AAATTT ACTAGT GGCTAGGAGGAATTACATATG) and Kdc_rev_Ec (AAATTT AAGCTT ATTACTTGTTCTGCTCCGCAAAC). The aminotransferase fragments were digested with KpnI/SpeI and the decarboxylase fragment was digested with SpeI/HindIII. Both fragments were ligated to KpnI/HindIII digested pBBR-lac to obtain the vectors pAKP-96 (vfl-kdcA (VVT)) (SEQ ID NO:9), pAKP-405 (=pAKP96 (vfl-kdcA (F382R))), pAKP-409 (=pAKP96 (vfl-kdcA (Q377I))), pAKP-411 (=pAKP96 (vfl-kdcA (Q377L))).

For E. coli optimized genes encoding the homoaconitase small subunit (AksE, SEQ ID NO:203 in WO 2010/104390), homoaconitase large subunit (AksD, SEQ ID NO:191 in WO 2010/104390) from M. aeolicus and homoisocitrate dehydrogenase from M. maripaludis (AksF, SEQ ID NO:221 in WO 2010/104390) were made synthetically (Geneart, Regensburg, Germany) together with the wild-type nifV gene (SEQ ID NO:149 in WO 2010/104390, M17349, Beynon, J., A. Ally, M. Cannon, F. Cannon, M. Jacobson, V. Cash and D. Dean. 1987. Comparative organization of nitrogen fixation-specific genes from Azotobacter vinelandii and Klebsiella pneumoniae: DNA sequence of the nifUSV genes. J. Bacteriol. 169(9):4024-9). In the optimization procedure internal restriction sites were avoided and common restriction sites were introduced at the start and stop to allow subcloning in expression vectors. Also, upstream of AksD the sequence of the tac promoter from pMS470 was added. Each ORF was preceded by a consensus ribosomal binding site and leader sequence to drive transcription and translation in E. coli. A synthetic AksA/AksF cassette was cut with NdeI/XbaI and a synthetic AksD/AksE cassette was cut with XbaI/HindIII. Fragments containing Aks genes were inserted in the NdeI/HindIII sites of pMS470 to obtain the vector used (SEQ ID NO:10). This plasmid was co-transformed with the plasmids pAKP-96 (vfl-kdcA (WT)) (SEQ ID NO:9), pAKP-405 (=pAKP96 (vfl-kdcA (F382R))), pAKP-409 (=pAKP96 (vfl-kdcA (Q3771))), pAKP-411 (=pAKP96 (vfl-kdcA (Q377L))), to E. coli strain BL21 to obtain the strains eAKP491, eAKP491_KdcA (F382R), eAKP491_KdcA (Q3771) and eAKP491_KdcA (Q377L).

Protein Expression and Metabolite Production in E. Coli

All Plasmids were transformed to E. coli BL21 for expression. Starter cultures were grown overnight in tubes with 10 ml 2*TY medium. 200 μl culture was transferred to shake flasks with 20 ml 2*TY medium. Flasks were incubated in an orbital shaker at 30° C. and 280 rpm. After 4 h IPTG was added at a final concentration of 0.1 mM and flasks were incubated for 16 h at 30° C. and 120 rpm. Cells from 20 ml culture were collected by centrifugation and resuspended in 4 ml M9 medium with 0.5% glucose in 24 well plates. After incubation for 48 h at 30° C. and 210 rpm cells were collected by centrifugation and supernatant was diluted 1:25 times in water and stored at −20° C. for analysis.

Method for the Determination of 6-ACA, AAP and Adipate

A Waters HSS T3 column 1.8 μm, 100 mm*2.1 mm was used for the separation of 6-ACA, AAP and adipate with gradient elution as depicted in Table 3. Eluens A consists of LC/MS grade water, containing 0.1% formic acid, and eluens B consists of acetonitrile, containing 0.1% formic acid. The flow-rate was 0.25 ml/min and the column temperature was kept constant at 40° C.

TABLE 3
gradient elution program used for the
separation of 6-ACA, AAP and adipate

| Time (min) | 0 | 5.0 | 5.5 | 10 | 10.5 | 15 |
|---|---|---|---|---|---|---|
| % A | 100 | 85 | 20 | 20 | 100 | 100 |
| % B | 0 | 15 | 80 | 80 | 0 | 0 |

A Waters micromass Quattro micro API was used in electrospray either positive or negative ionization mode, depending on the compounds to be analyzed, using multiple reaction monitoring (MRM). The ion source temperature was kept at 130° C., whereas the desolvation temperature is 350° C., at a flow-rate of 500 L/h r.

For adipate the deprotonated molecule was fragmented with 10-14 eV, resulting in specific fragments from losses of e.g. H₂O, CO and CO₂.

For 6-ACA and AAP the protonated molecule was fragmented with 13 eV, resulting in specific fragments from losses of H₂O, NH₃ and CO.

To determine concentrations, a calibration curve of external standards of synthetically prepared compounds was run to calculate a response factor for the respective ions. This was used to calculate the concentrations in samples. Samples were diluted appropriately (2-10 fold) in eluent A to overcome ion suppression and matrix effects.

To determine concentrations a standard curve of synthetically prepared compounds was run to calculate a response factor for the respective ions. This was used to calculate the concentrations in unknown samples.

Analysis of Supernatant

TABLE 4
6-ACA, AAP and Adipate production in M9 medium using
strains with various decarboxylases.

| | 6-ACA | Adipate | |
|---|---|---|---|
| strain | (mg/l) | (mg/l) | AAP (mg/l) |
| eAKP491 | 15 | 114 | 132 |
| eAKP491_KdcA | 19 | 113 | 85 |
| (F382R) | | | |
| eAKP491_KdcA | 27 | 187 | 43 |
| (Q377I) | | | |
| eAKP491_KdcA | 21 | 137 | 81 |
| (Q377L) | | | |

Supernatant was diluted 25 times with water prior to UPLC-MS/MS analysis. Results, shown in Table 4, clearly show that the level of 6-ACA in the E. coli strains eAKP491_KdcA (F382R), eAKP491_KdcA (Q3771) and eAKP491_KdcA (Q377L) is significantly higher as compared to the control strain eAKP491 showing the superior performance of the KdcA variants tested.

### EXAMPLE 4

### Testing KdcA Variants In Vivo for Improved 6-ACA Production in C. glutamicum

Cloning of the Genes

Protein sequences for the Methanococcus aeolicus Nankai 3 homoaconitase small subunit (AksE, Maeo_{—}0652 [SEQ ID NO:204 in WO 2010/104390, Protein ID YP_{—}001324848]), homoaconitase large subunit (AksD, Maeo_{—}0311, [SEQ ID NO:192 in WO 2010/104390, Protein ID YP_{—}001324511]), homoisocitrate dehydrogenase (AksF, Maeo_{—}1484 [SEQ ID NO:219 in WO 2010/104390, Protein ID YP_{—}001325672]), the A. vinelandii homocitrate synthase (NifV, [SEQ ID NO:75 in WO 2010/104390, Protein ID P05342]), the aminotransferase protein from Vibrio fluvialis JS17 (SEQ ID NO:2 in WO 2010/104390) and the Lactococcus lactis branched chain alpha-keto acid decarboxylase KdcA (SEQ ID NO:2) were retrieved from databases.

All genes were codon pair optimized. The gene encoding the aminotransferase gene from Vibrio fluvialis JS17 Vfl (SEQ ID NO:3 in WO 2010/104390), the gene coding for the Lactococcus lactis branched chain alpha-keto acid decarboxylase KdcA (SEQ ID NO:3), the gene encoding the homoaconitase small subunit AksE (SEQ ID NO:203 in WO 2010/104390) from M. aeolicus and the homoaconitase large subunit AksD (SEQ ID NO:191 in WO 2010/104390) from M. aeolicus were optimized for E. coli whereas the gene encoding the homoisocitrate dehydrogenase AksF (SEQ ID NO:7) from M. aeolicus and the gene encoding the A. vinelandii homocitrate synthase nifV (SEQ ID NO:8) were optimized for C. glutamicum. Sequences were made synthetically (Geneart, Regensburg, Germany). In the optimization procedure internal restriction sites were avoided and common restriction sites were introduced at the start and stop to allow subcloning in expression vectors. Also, each ORF was preceded by a consensus ribosomal binding site and leader sequence to drive transcription and translation.

Vectors pAKP-96 (vfl-kdcA (WT)) (SEQ ID NO:9), pAKP-405, pAKP-407, pAKP-409, pAKP-411 (Example 3) were digested with EcoR1/HinD3 and the fragments containing the vlf-kdcA gene were isolated from gel using the Zymoclean Gel DNA Recovery kit, (Zymo Research corp. Irvine, Calif. 92614, U.S.A.). Subsequently the fragment was made blunt using End-It DNA End-Repair kit (Epicentre Biotech. Madison, Wis. 53713, USA). To clone this fragment into the E. coli-Corynebacterium shuttle vector pVWEx1 (Peters-Wendisch, P. G., B. Schiel, V. F. Wendisch, E. Katsoulidis, B. Mockel, H. Sahm, and B. J. Eikmanns. 2001. Pyruvate carboxylase is a major bottleneck for glutamate and lysine production by Corynebacterium glutamicum. J. Mol. Microbiol. Biotechnol. 3:295-300), this vector was digested with xba1, made blunt with the End-It DNA End-Repair kit and treated with shrimp alkaline phosphatase according to the suppliers instructions yielding the vectors pAKP-453 (vfl-kdcA (WT)), pAKP-502 (vfl-kdcA (F382R)), pAKP-503 (vfl-kdcA (L261G)), pAKP-504 (vfl-kdcA (Q377I)), pAKP-505 (vfl-kdcA (Q377L)).

A synthetic AksA/AksF cassette was cut with NdeI/XbaI and a synthetic AksD/AksE cassette was cut with XbaI/HindIII. Fragments containing Aks genes were inserted in the NdeI/HindIII sites of the E. coli-Corynebacterium shuttle vectors pEKEx3 yielding plasmid pAKP-485 (SEQ ID NO:11). This plasmid was co-transformed with the plasmids pAKP-453 (vfl-kdcA (WT)), pAKP-502 (vfl-kdcA (F382R)), pAKP-503 (vfl-kdcA (L261G)), pAKP-504 (vfl-kdcA (Q377I)), pAKP-505 (vfl-kdcA (Q377L).

Protein Expression and Metabolite Production in C. Glutamicum

All Plasmids were transformed to wild-type C. glutamicum strain ATCC13032 for expression. Starter cultures were grown overnight in tubes with 10 ml 2×TY medium+0.5% glucose. 300 μl culture was transferred to shake flasks with or without baffle with 30 ml 2×TY medium and 1 mM IPTG. Flasks were incubated in an orbital shaker at 30° C. and 120 rpm. Flasks were incubated 20 h at 30° C. and 15 ml of cell culture was collected by centrifugation and resuspended in 5 ml YSTB medium (consisting of (per liter) 8.37 g of 3-[N-morpholino]-propanesulfonic acid (MOPS), 0.72 g of N-tris[hydroxymethyl]-methylglycine (Tricine), 4.05 g of NH₄Cl, 1 g of KCl, 0.3 g of K₂HPO₄, 0.23 g of MgCl₂.6H₂O, 50 mg of CaCl₂.2H₂O, 0.2 g of EDTA, 50 mg of K₂SO₄, 4.5 mg of ZnSO₂.7H₂O, 0.3 mg of CoCl₂.6H₂0, 1 mg of MnCl₂.4H₂O, 0.3 mg of CuSO₄.5H₂O, 4.5 mg of CaCl₂.2H₂0, 3 mg of FeSO₄.7H₂O, 0.4 mg of NaMo0₄.2H₂0, 1 mg of H₃BO₃, 0.1 mg of KI, 0.05 mg of biotin, 1 mg of calcium pantothenate, 1 mg of Nicotinic acid, 25 mg of inositol, 1 mg of thiamine HCl, 1 mg of pyridoxine HCl and 0.2 mg of para-aminobenzoic acid) with 0.1M Acetate and 0.5% glucose in 24 well plates. After incubation for 96 h at 30° C. and 200 rpm cells were collected by centrifugation and pellet and supernatant were separated and stored at −20 C for analysis.

Preparation of Samples for Analytics

Both extracellular (supernatant) and intracellular (cell extract) fraction were analyzed for the presence of products. Culture supernatant was directly analyzed after 1:5 times or 1:25 times dilution in water. For the preparation of cell extracts, cells from small scales growth (see previous paragraph) were harvested by centrifugation. The cell pellets were resuspended in 1 ml of 100% ethanol and vortexed vigorously. The cell suspension was heated for 2 min at 95° C. and cell debris was removed by centrifugation. The supernatant was evaporated in a vacuum dryer and the resulting pellet was dissolved in 200 μl deionized water. Remaining debris was removed by centrifugation and the supernatant was stored at −20° C.

Analysis of Supernatant

Supernatant was diluted 5 times with water prior to UPLC-MS/MS analysis (see Example 3). Results, shown in Table 5, clearly show that using the conditions described the strain containing the wild-type KdcA did not accumulate any detectable 6-ACA or Adipate in the supernatants. Strains expressing the KdcA variants with the improved specificity for AKP now accumulate significant amounts of 6-ACA and Adipate in the supernatants showing the superior performance of the KdcA variants with improved specificity for AKP over the wild-type KdcA. From this table it is clear that mutants with a reduced conversion rate, as compared to the wild-type KdcA, also have a beneficial effect on the amount of 6-ACA produced in vivo.

TABLE 5
6-ACA and Adipate production in C. glutamicum strains with various
decarboxylases.

| | | 6-ACA | Adipate |
|---|---|---|---|
| plasmids | Decarboxylase | (mg/l) | (mg/l) |
| pAKP-485/pAKP-453 | KdcA WT | n.d | n.d |
| pAKP-485/pAKP-502 | KdcA | 7.3 | 10.5 |
| | (F382R) | | |
| pAKP-485/pAKP-503 | KdcA | 4.2 | 3.4 |
| | (L261G) | | |
| pAKP-485/pAKP-504 | KdcA | 15.6 | 13.0 |
| | (Q377I) | | |
| pAKP-485/pAKP-505 | KdcA | 1.5 | 1.2 |
| | (Q377L) | | |

(n.d. = not detected)

### EXAMPLE 5

### Design and Screening of Combinatorial KdcA Libraries

Based on the results in example 2, four combinatorial libraries were designed:
1. L261G, Q377LIV, F382RE, M538GAS, F542DCSI, N546P and K547P
2. R382, M538X and F542X
3. L261GAYD, Q377MILV, R382ECS, M538ACSWG, F542ILVDCSA, N546P and K547P
4. F382RKQN, V461ILF, I465LVASN, L535VIFA and F542RKQN

For library 1 the following 7 amino acid positions were included: 261, 377, 382, 538, 542, 546 and 547. These positions contain amino acids L261, Q377, F382, M538, F542, N546 and K547 which represent wild type KdcA. In the context of the combinatorial libraries 1 to 4, the wording L261G indicates that besides the wild type amino acid L, also amino acid G is allowed at position 261, Q277LIV indicates that besides the wild type amino acid Q also amino acids L, I and V are allowed at position 277, F382RE indicates that at position 382 besides the wild type amino acid F also amino acids R and E are allowed, and so on. A wild type bias was used in order to obtain combinatorial mutants which contain on the average 3 amino acid substitutions per mutant. This was verified by sequencing a limited number of genes from the library e.g. 50. For library 2, mutant F382R was taken as the starting sequence for saturation mutagenesis at positions 538 and 542. So R382 was fixed. X indicates that at positions 538 and 542 all 20 amino acids are allowed, which results in 400 possible mutants. Library 3 is very similar to library 1 with respect to the positions which are substituted, but the number of amino acids that is allowed is increased and the starting sequence is mutant F382R as was also used for library 2. Contrary to library 2 now R382 is not fixed. Besides R also amino acids E, C and S are allowed at position 382. Finally library 4 comprises 5 positions which are subjected to substitution by the amino acids as indicated.

Libraries 1 to 4 were constructed by Sloning BioTechnology GmbH (Zeppelinstrasse 4, Puchheim, 82178 Germany) using their Slonomics® technology and were introduced into the pBAD-kdcA vector. Mutations were made in the context of the gene which was optimized for expression in E. coli (SEQ ID NO:3). The libraries were subsequently cloned into the pBAD/Myc-His-DEST expression vector using the Gateway technology (Invitrogen) via the introduced attB sites and pDONR201 (Invitrogen) as entry vector as described in the manufacturer's protocols (www.invitrogen.com). The corresponding expression strains were obtained by transformation of chemically competent E. coli TOP10 (Invitrogen) with the respective pBAD-expression vectors containing the respective libraries

The expression libraries were plated and grown on Q-trays. For each library about 1000 clones were picked using the Q-pix to inoccuate half-deep-well plates (Westburg/Thermo) containing 500 μl/well 2* TY medium (16 gr/l tryptone, 10 gr/l yeast extract, 5 gr/l NaCl) with 100 ug/mlampicillin. Growing the clones, preparation of the cell free extracts and testing for improved activity and specificity was done as described in example 1. Instead of incubating the main culture for 7 hours at 30 dgrC, the incubation time was extended to 30 hours. Finally the 96 best clones observed during testing were retested and sequenced. Wild type KdcA was always included as a reference. Of the 96 clones retested the 32 best performing clones are shown in the table below (Table 6) with the amino acid substitutions observed with respect to the wild type enzyme (SEQ ID NO:2).

TABLE 6
Results obtained with combinatorial KdcA libraries

| | ratio | 5FVA (mM) | AKP conversion |
|---|---|---|---|
| | 5-FVA/4-FBA | after 16 hours | (%) |
| Mutations | after 16 hours | conversion | after 16 hours |
| wt | 1.13 | 14.46 | 57.85 |
| L261G, Q377V, M538W, N546T, K547P | 12.91 | 18.81 | 75.26 |
| L261Y, Q377V, F382R, F542L, K547P | 19.25 | 16.40 | 65.59 |
| L261Y, Q377V, F382R, F542S | >>200 | 12.77 | 51.06 |
| L261Y, Q377V, F382R | >>200 | 11.04 | 44.18 |
| L261D, Q377I, F382R, F542S | >>200 | 7.31 | 29.26 |
| L261D, Q377V, F382R, F542C, N546P | >>200 | 5.81 | 23.26 |
| L261G, Q377I | 17.19 | 21.55 | 86.20 |
| L261G, Q377V | 10.93 | 20.75 | 82.98 |
| L261G, Q377L | >>200 | 7.59 | 30.38 |
| Q377V, F382R, F542L | 43.53 | 24.38 | 97.54 |
| Q377L, F382R, M538A, F542L | >>200 | 23.74 | 94.98 |
| Q377V, F382R, F542I, K547P | >>200 | 15.69 | 62.75 |
| Q377I, F382S, M538S | 7.15 | 13.23 | 52.91 |
| Q377V, F382R, F542V | >>200 | 11.96 | 47.85 |
| Q377I, F382R | >>200 | 10.92 | 43.70 |
| Q377V, F382R, M538S, K547P | >>200 | 7.45 | 29.79 |
| Q377L, F382S | >>200 | 2.98 | 11.93 |
| Q377V, F382R, F542I | 135.69 | 23.14 | 92.54 |
| Q377V, F382R, M538A | >>200 | 10.26 | 41.04 |
| Q377V, M538A | 5.24 | 27.11 | 108.45 |
| Q377L, M538G | 11.46 | 20.82 | 83.29 |
| Q377I, M538A | 8.28 | 25.61 | 102.43 |
| Q377I, F542I | 7.33 | 24.73 | 98.94 |
| Q377L, N546P | 24.21 | 21.61 | 86.42 |
| Q377I, K547P | 203.36 | 18.46 | 73.83 |
| F382N, V461I, L535A | 3.11 | 25.77 | 103.10 |
| F382R, M538L, F542W | 7.46 | 27.31 | 109.25 |
| F382R, M538W | 14.82 | 25.40 | 101.60 |
| F382R, F542M | 17.31 | 26.24 | 104.96 |
| F382R, N546P | 78.03 | 20.98 | 83.93 |
| M538S, N546H | 1.33 | 29.47 | 117.89 |
| M538W, K547P | 27.40 | 24.37 | 97.49 |

An 5-FVA/4-FBA ratio >>200 indicates that the amount of FVA produced was very close or similar to the total amount of aldehyde observed. A ratio 5-FVA/4-FBA of 200 was about the highest ratio that could be determined in respect to the detection limit for 4-FBA.

1. An alpha-ketopimelic acid decarboxylase enzyme having at least 50% sequence identity with SEQ ID NO:2, wherein the enzyme comprises at least one mutation selected from the group of substitutions corresponding to 072L, 072M, 101D, 101E, 101F, 101L, 104D, 104Q, 104W, 111M, 166K, 166R, 240A, 240G, 241L, 241N, 241R, 258R, 261A, 261D, 261G, 261W, 261Y, 284C, 284I, 284S, 284V, 290E, 290F, 290N, 290Q, 290Y, 291S, 377A, 377I, 377L, 377M, 377T, 377V, 381H, 382A, 382C, 382E, 382I, 382K, 382N, 382R, 382S, 382V, 382Y, 461I, 461 L, 461M, 461T, 465C, 465F, 465L, 465M, 532C, 532T, 534G, 535A, 535C, 535G, 535Q, 535S, 538A, 538C, 538G, 538H, 538L, 538S, 538W, 539H, 539L, 539Q, 539R, 539T, 541N, 541V, 542A, 542C, 542D, 542E, 542G, 542H, 542I, 542K, 542L, 542M, 542N, 542Q, 542R, 542S, 542T, 542V, 542W, 545C, 545D, 545E, 545F, 545K, 545R, 545S, 545T, 545V, 545W, 546A, 546E, 546F, 546G, 546H, 546P, 546T, 546V, 546W, 546Y and 547P in SEQ ID NO:2, with the provisio that if the enzyme has only one mutation compared to SEQ ID NO:2, that mutation is not 4611 or 538W in SEQ ID NO:2.

2. An alpha-ketopimelic acid decarboxylase enzyme according to claim 1, wherein the mutation is selected from the group of substitutions corresponding to 072L, 072M, 101D, 111M, 240A, 240G, 241L, 241R, 261A, 261G, 261Y, 284I, 290F, 290N, 377I, 377L, 377M, 382A, 382C, 382E, 382R, 382Y, 461I, 461 L, 461T, 534G, 535A, 535C, 535S, 538A, 538C, 539T, 541V, 542 I and 542L in SEQ ID NO:2.

3. An alpha-ketopimelic acid decarboxylase enzyme according to claim 1, wherein the enzyme comprises at least two mutations selected from the group of substitutions corresponding to 261A, 261D, 261G, 261Y, 377I, 377L, 377M, 377V, 382C, 382E, 382N, 382S, 382R, 538A, 538C, 538G, 538L, 538S, 538W, 542A, 542C, 542D, 542I, 542L, 542M, 542S, 542V, 542W, 546H, 546P, 546T and 547P in SEQ ID NO:2.

4. Nucleic acid encoding an alpha-ketopimelic acid decarboxylase enzyme according to claim 1.

5. A host cell comprising a gene encoding an alpha-ketopimelic acid decarboxylase enzyme according to claim 1.

6. A host cell according to claim 5, wherein the host cell is selected from the group of Aspergillus, Penicillium, Saccharomyces, Kluyveromyces, Pichia, Candida, Hansenula, Bacillus, Corynebacterium, and Escherichia.

7. A method for preparing 5-formylvaleric acid, comprising decarboxylating alpha-ketopimelic acid, wherein the decarboxylation is catalysed by an alpha-ketopimelic acid decarboxylase enzyme according to claim 1.

8. A method for preparing 6-aminocaproic acid, comprising preparing 5-formylvaleric acid in a method according to claim 7 and converting 5-formylvaleric acid into 6-aminocaproic acid.

9. Method according to claim 8, wherein the conversion of 5-formylvaleric acid is catalysed by an aminotransferases (E.C. 2.6.1) or an amino acid dehydrogenases (E.C.1.4.1).

10. A method for preparing caprolactam, comprising preparing 6-aminocaproic acid in a method according to claim 8 and cyclising 6-aminocaproic acid into caprolactam.

11. A method for preparing adipic acid, comprising preparing 5-formylvaleric acid in a method according to claim 7 and converting 5-formyl valeric acid into adipic acid.

12. A method for preparing 1,6-diaminohexane, comprising preparing adipic acid according to claim 11 and converting adipic acid into 1,6-diaminohexane.

13. A method for preparing 1,6-diaminohexane, comprising preparing 6-aminocaproic acid in a method according to claim 8 and converting 6-aminocaproic acid into 1,6-diaminohexane.
